(19) United States
(12) Patent Application Publication
(10) Pub. No.: US 2025/0186478 A1
(43) Pub. Date: Jun. 12, 2025

(54) METHODS AND COMPOSITIONS FOR TARGETED GENE TRANSFER

(71) Applicant: The University of North Carolina at Chapel Hill, Chapel Hill, NC (US)

(72) Inventors: Kenton Woodard, Carrboro, NC (US); Richard Jude Samulski, Chapel Hill, NC (US)

(21) Appl. No.: 19/042,219

(22) Filed: Jan. 31, 2025

Related U.S. Application Data
(63) Continuation of application No. 16/326,126, filed on Feb. 15, 2019, now abandoned.
(60) Provisional application No. 62/375,666, filed on Aug. 16, 2016.

(51) Int. Cl. A61K 31/7088; A61K 48/00; A61P 27/00; A61P 27/02; C07K 14/005; C12N 15/63; C12N 15/86

(52) U.S. Cl. CPC A61K 31/7088; A61K 48/00; A61P 27/00; A61P 27/02; C07K 14/005; C12N 15/63; C12N 15/86; C12N 2750/14122; C12N 2750/14123; C12N 2750/14142; C12N 2750/14143

(57) ABSTRACT

The present invention provides AAV capsid proteins comprising a modification in the amino acid sequence and virus capsids and virus vectors comprising the modified AAV capsid protein. The invention also provides methods of administering the virus vectors and virus capsids of the invention to a cell or to a subject in vivo.

### STATEMENT OF PRIORITY

This application is a continuation of U.S. application Ser. No. 16/326,126, filed Feb. 15, 2019, which is a 35 U.S.C. § 371 national phase application of International Application Serial No. PCT/US2017/047123, filed Aug. 16, 2017, which claims the benefit, under 35 U.S.C. § 119 (e), of U.S. Provisional Application No. 62/375,666, filed Aug. 16, 2016, the entire contents of each of which are incorporated by reference herein.

### STATEMENT OF GOVERNMENT SUPPORT

This invention was made with government support under Grant Number DK084033 awarded by the National Institutes of Health. The government has certain rights in the invention.

### STATEMENT REGARDING ELECTRONIC FILING OF A SEQUENCE LISTING

A Sequence Listing in XML format, entitled 5470-778CT_ST26.xml, 20,343 bytes in size, generated on Jan. 29, 2025, and filed herewith, is hereby incorporated by reference in its entirety for its disclosure.

### FIELD OF THE INVENTION

The present invention relates to modified capsid proteins from adeno-associated virus (AAV) and virus capsids and virus vectors comprising the same. In particular, the invention relates to modified AAV capsid proteins and capsids comprising the same that can be incorporated into virus vectors to confer a desirable transduction profile with respect to a target tissue of interest.

### BACKGROUND OF THE INVENTION

New adeno-associated virus (AAV) strains isolated from animal tissues and adenoviral stocks have expanded the panel of AAV vectors available for therapeutic gene transfer applications. Comprehensive efforts to map tissue tropisms of these AAV isolates in animal models are currently underway. The ability to direct homing of AAV vectors to selective organs is useful for gene therapy and other therapeutic applications.

Adeno-associated virus (AAV) has become the vector of choice for viral gene transfer and has shown great promise in clinical trials. Of importance is the successful treatment of the retina by subretinal delivery. Development of a less invasive injection route is met by intravitreal delivery, but delivery of AAV by this route results in poor transduction outcomes. The inner limiting membrane (ILM) creates a barrier separating the vitreous and the retina.

The present invention addresses a need in the art for nucleic acid delivery vectors with desirable targeting features.

### SUMMARY OF THE INVENTION

The present invention provides a method of introducing a nucleic acid molecule into a cell of a retina and/or retinal pigment epithelium of a subject, comprising intravitreally administering an adeno-associated virus (AAV) serotype 4 (AAV4) vector comprising an AAV4 capsid protein, wherein the AAV4 capsid protein comprises a substitution at amino acid residue K530 and/or further comprises a substitution at one or more of amino acid residues S584, N585, S586 and N587 in any combination, wherein the numbering of the residues is based on the amino acid sequence of SEQ ID NO:4 (amino acid sequence of AAV4 capsid protein).

The present invention also provides a method of introducing a nucleic acid molecule into a cell of a retina and/or retinal pigment epithelium of a subject, comprising intravitreally administering an adeno-associated virus (AAV) serotype 5 (AAV5) vector comprising an AAV5 capsid protein, wherein the AAV5 capsid protein comprises a substitution at amino acid residue K517 and/or further comprises a substitution at one or more of amino acid residues S575, S576, T577 and T578 in any combination, wherein the numbering of the residues is based on the amino acid sequence of SEQ ID NO:5 (amino acid sequence of AAV5 capsid protein).

Additionally provided herein is a method of introducing a nucleic acid molecule into a cell of a retina and/or retinal pigment epithelium of a subject, comprising intravitreally administering an adeno-associated virus (AAV) serotype 7 (AAV7) vector comprising an AAV7 capsid protein, wherein the AAV7 capsid protein comprises a substitution at amino acid residue K533 and/or further comprises a substitution at one or more of amino acid residues A587, A588, N589 and R590 in any combination, wherein the numbering of the residues is based on the amino acid sequence of SEQ ID NO:6 (amino acid sequence of AAV7 capsid protein).

The present invention further provides a method of introducing a nucleic acid molecule into a cell of a retina and/or retinal pigment epithelium of a subject, comprising intravitreally administering an adeno-associated virus (AAV) serotype 8 (AAV8) vector comprising an AAV8 capsid protein, wherein the AAV8 capsid protein comprises a substitution at amino acid residue K533 and/or further comprises a substitution at one or more of amino acid residues Q587, Q588, N589 and T590 in any combination, wherein the numbering of the residues is based on the amino acid sequence of SEQ ID NO:7 (amino acid sequence of AAV8 capsid protein).

Also provided herein is a method of introducing a nucleic acid molecule into a cell of a retina and/or retinal pigment epithelium of a subject, comprising intravitreally administering an adeno-associated virus (AAV) serotype 9 (AAV9) vector comprising an AAV9 capsid protein, wherein the AAV9 capsid protein comprises a substitution at amino acid residue K531 and/or further comprises a substitution at one or more of amino acid residues Q587, A588, N589 and T 590 in any combination, wherein the numbering of the residues is based on the amino acid sequence of SEQ ID NO: 8 (amino acid sequence of AAV9 capsid protein).

Furthermore, the present invention provides a method of treating a disorder or defect of the eye in a subject, comprising intravitreally administering to the subject the virus vector of this invention, wherein the virus vector comprises a nucleic acid molecule that encodes a therapeutic protein or therapeutic DNA effective in treating the disorder or defect of the eye in the subject.

These and other aspects of the invention are addressed in more detail in the description of the invention set forth below.

### BRIEF DESCRIPTION OF THE DRAWINGS

FIG. 1. Green fluorescence protein (GFP) fluorescence following intravitreal delivery of rAAV2 vector and its HS-binding deficient variants twelve weeks post-injection. Quantification of fundus images showed a 300-fold decrease in expression between rAAV2 (HS-binding) and rAAV2i8 (ablated HS-binding). Immunohistochemistry (IHC) of rAAV2-injected retinas shows fluorescence mainly in the RGC with fewer GFP-positive somas in the INL. Graph is shown with error bars indicating the standard error mean (SEM) and significance is detected by a non-parametric t-test (**p<0.01).

FIG. 2. Schematic of the retina depicts the trafficking of rAAV following intravitreal delivery.

FIG. 3. qPCR analysis of viral binding to human retinas ex vivo. Results are quantified as vector genomes per cell genome. rAAV2 (HS-binding) vector shows the greatest presence at the retina with few transgenes found elsewhere. The presence of transgenes delivered by rAAV2i8 (ablated HS-binding) were low in all collected tissues but showed a significant increase compared to rAAV2-delivered transgenes in both the choroid and sclera. Error bars indicated standard deviation.

FIG. 4. GFP fluorescence following intravitreal delivery of HS-binding variants of rAAV1 eight weeks post-injection. Quantification of fundus images shows a 3-fold increase with rAAV1-E531K (HS-binding) capsid compared to rAAV1 (non HS-binding) capsid. Graph is shown with error bars indicating the SEM and significance by a non-parametric t-test (*p<0.05).

FIG. 5. Chimeric capsids suggest tropism is influenced by other motifs other than HS binding. Elements of rAAV1 were applied to rAAV2 using the chimeric rAAV2.5 capsid and imaged for intravitreal delivery. Quantification of the fundus fluorescence for the collection of capsids. Error bars represent the SEM.

FIG. 6. In vitro competition assay using soluble heparin to block the transduction of rAAV of HEK293 cells. Viruses were incubated with increasing doses of soluble heparin and applied to cell culture at a multiplicity of infection of 10,000 vg per cell. rAAV2 displayed a dose-dependent decrease in transduction which was not observed with either rAAV1 or rAAV1-E531K. The amount of transduction of rAAV1-E531K was lower than rAAV1 in all conditions. Error bars shown as SEM.

### DETAILED DESCRIPTION OF THE INVENTION

The present invention will now be described with reference to the accompanying drawings, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

The present invention is based on the unexpected discovery that intravitreal transduction of cells of the retina and/or retinal pigment epithelium can be enhanced by the addition of the heparan sulfate binding motif on the AAV capsid. Thus, in one embodiment, the present invention provides a method of introducing a nucleic acid molecule into a cell of a retina and/or retinal pigment epithelium of a subject, comprising intravitreally administering an adeno-associated virus (AAV) serotype 4 (AAV4) vector comprising an AAV4 capsid protein, wherein the AAV4 capsid protein comprises a substitution at amino acid residue K530 and/or further comprises a substitution at one or more of amino acid residues S584, N585, S586 and N587 in any combination, wherein the numbering of the residues is based on the amino acid sequence of SEQ ID NO:4 (amino acid sequence of AAV4 capsid protein).

The present invention also provides a method of introducing a nucleic acid molecule into a cell of a retina and/or retinal pigment epithelium of a subject, comprising intravitreally administering an adeno-associated virus (AAV) serotype 5 (AAV5) vector comprising an AAV5 capsid protein, wherein the AAV5 capsid protein comprises a substitution at amino acid residue K517 and/or further comprises a substitution at one or more of amino acid residues S575, S576, T577 and T578 in any combination, wherein the numbering of the residues is based on the amino acid sequence of SEQ ID NO:5 (amino acid sequence of AAV5 capsid protein).

Additionally provided herein is a method of introducing a nucleic acid molecule into a cell of a retina and/or retinal pigment epithelium of a subject, comprising intravitreally administering an adeno-associated virus (AAV) serotype 7 (AAV7) vector comprising an AAV7 capsid protein, wherein the AAV7 capsid protein comprises a substitution at amino acid residue K533 and/or further comprises a substitution at one or more of amino acid residues A587, A588, N589 and R590 in any combination, wherein the numbering of the residues is based on the amino acid sequence of SEQ ID NO:6 (amino acid sequence of AAV7 capsid protein).

The present invention further provides a method of introducing a nucleic acid molecule into a cell of a retina and/or retinal pigment epithelium of a subject, comprising intravitreally administering an adeno-associated virus (AAV) serotype 8 (AAV8) vector comprising an AAV8 capsid protein, wherein the AAV8 capsid protein comprises a substitution at amino acid residue K533 and/or further comprises a substitution at one or more of amino acid residues Q587, Q588, N589 and T590 in any combination, wherein the numbering of the residues is based on the amino acid sequence of SEQ ID NO:7 (amino acid sequence of AAV8 capsid protein).

Also provided herein is a method of introducing a nucleic acid molecule into a cell of a retina and/or retinal pigment epithelium of a subject, comprising intravitreally administering an adeno-associated virus (AAV) serotype 9 (AAV9) vector comprising an AAV9 capsid protein, wherein the AAV9 capsid protein comprises a substitution at amino acid residue K531 and/or further comprises a substitution at one or more of amino acid residues Q587, A588, N589 and T 590 in any combination, wherein the numbering of the residues is based on the amino acid sequence of SEQ ID NO:8 (amino acid sequence of AAV9 capsid protein).

Furthermore, the present invention provides a method of treating a disorder or defect of the eye in a subject, comprising intravitreally administering to the subject the virus vector of this invention, wherein the virus vector comprises a nucleic acid molecule that encodes a therapeutic protein or therapeutic DNA effective in treating the disorder or defect of the eye in the subject.

In further embodiments, the methods of this invention can be carried out with an AAV vector comprising a capsid protein that has been modified as described below. Specifically, in some embodiments, the heparan sulfate binding motif (AAV2 numbering: 484R, 485Q, 486Q, 487R, 488V, 489S, 490K, 491T, 527K, 528D, 529D, 530E, D531E, 532K, S585R, S586G, S587N, T588R) can be graphed onto an AAV1 capsid protein and/or the AAV1 capsid protein can comprise Tyr mutations (AAV2 numbering: Y252F, Y272F, Y444F, Y500F, Y700F, Y704F, Y730F,), galactose motif (AAV2 numbering: G469N, 470M, S471A, 472V, P474G, 500F) insertion of peptide with the amino acid abbreviation LALGETTRPA (AAV2 numbering: 587), or deletion mutation (AAV2 numbering: T265), in any combination. Amino acid residue numbering is based on the amino acid sequence of AAV2 (SEQ ID NO:2).

In some embodiments, the heparan sulfate binding motif (AAV2 numbering: 484R, 485Q, 486 Q, 487 R, 488 V, 489 S, 490 K, 491T, 527K, 528D, 529D, 530E, 531E, 532K, 585R, 586G, 587N, 588R) can be graphed onto an AAV2 capsid protein and/or the AAV2 capsid protein can comprise Tyr mutations (AAV2 numbering: Y252F, Y272F, Y444F, Y500F, Y700F, Y704F, Y730F), galactose motif (AAV2 numbering: D469N, 1470M, R471A, D472V, S474G, Y500F), insertion of peptide with the amino acid abbreviation LALGETTRPA (AAV2 numbering: 587), or insertion mutation (AAV2 numbering: 265), in any combination. Amino acid residue numbering is based on the amino acid sequence of AAV2 (SEQ ID NO:2).

In some embodiments, the heparan sulfate binding motif (AAV2 numbering: 484R, 485Q, 486Q, 487R, L488V, 489S, 490K, 491T, 527K, 528D, 529D, 530E, 531E, 532K, S585R, S586G, N587N, T588R) can be graphed onto an AAV3B capsid protein and/or the AAV3B capsid protein can comprise Tyr mutations (AAV2 numbering: Y252F, Y272F, Y444F, Y500F, Y700F, Y704F, Y730F,), galactose motif (AAV2 numbering: S469N, 470M, S471A, N472V, A474G, 500F) insertion of peptide with the amino acid abbreviation LALGETTRPA (AAV2 numbering: 587), or insertion mutation (AAV2 numbering: 265), in any combination. Amino acid residue numbering is based on the amino acid sequence of AAV2 (SEQ ID NO:2).

In some embodiments, the heparan sulfate binding motif (AAV2 numbering: K484R, 485Q, 486Q, G487R, F488V, 489S, 490K, 491T, G527K, P528D, A529D, D530E, S531E, 532K, S585R, N586G, S587N, N588R) can be graphed onto an AAV4 capsid protein and/or the AAV4 capsid protein can comprise Tyr mutations (AAV2 numbering: Y252F, Y272F, Y444F, Y500F, Y700F, Y704F, Y730F), galactose motif (AAV2 numbering: 469N, F470M, S471A, N472V, K474G, S500F), insertion of peptide with the amino acid abbreviation LALGETTRPA (AAV2 numbering: 587), or insertion mutation (AAV2 numbering: 265), in any combination. Amino acid residue numbering is based on the amino acid sequence of AAV2 (SEQ ID NO:2).

In some embodiments, the heparan sulfate binding motif (AAV2 numbering: 484R, T485Q, G487R, W488V, N489S, L490K, G491T, L527K, Q528D, G529D, S530E, N531E, T532K, S585R, S586G, T587N, T588R) can be graphed onto an AAV5 capsid protein and/or the AAV5 capsid protein can comprise Tyr mutations (AAV2 numbering: Y252F, Y272F, Y444F, Y500F, Y700F, Y704F, Y730F), galactose motif (AAV2 numbering: R469N, Y470M, 471A, N472V, Y474G, S500F), insertion of peptide with the amino acid abbreviation LALGETTRPA (AAV2 numbering: 587), or insertion mutation (AAV2 numbering: 265), in any combination. Amino acid residue numbering is based on the amino acid sequence of AAV2 (SEQ ID NO:2).

In some embodiments, the heparan sulfate binding motif (AAV2 numbering: 484R, 485Q, 486Q, 487R, 488V, 489S, 490K, 491T, 527K, 528D, 529D, 530E, 531E, R532K, A585R, A586G, 587N, T588R) can be graphed onto an AAV7 capsid protein and/or the AAV7 capsid protein can comprise Tyr mutations (AAV2 numbering: Y252F, Y272F, Y444F, Y500F, Y700F, Y704F, Y730F,), galactose motif (AAV2 numbering: T469N, 470M, 471A, E472V, A474G, 500F) insertion of peptide with the amino acid abbreviation LALGETTRPA (AAV2 numbering: 587), or insertion mutation (AAV2 numbering: 265), in any combination. Amino acid residue numbering is based on the amino acid sequence of AAV2 (SEQ ID NO:2).

In some embodiments, the heparan sulfate binding motif (AAV2 numbering: 484R, 485Q, 486Q, 487R, 488V, 489S, T490K, 491T, 527K, 528D, 529D, 530E, 531E, R532K, Q585R, Q586G, N587N, T588R) can be graphed onto an AAV8 capsid protein and/or the AAV 8 capsid protein can comprise Tyr mutations (AAV2 numbering: Y252F, Y272F, Y444F, Y500F, Y700F, Y704F, Y730F,), galactose motif (AAV2 numbering: T469N, 470M, 471A, N472V, A474G, 500F) insertion of peptide with the amino acid abbreviation LALGETTRPA (AAV2 numbering: 587), or substitution mutation (AAV2 numbering: S265), in any combination. Amino acid residue numbering is based on the amino acid sequence of AAV2 (SEQ ID NO:2).

In some embodiments, the heparan sulfate binding motif (AAV2 numbering: 484R, 485Q, 486Q, 487R, 488V, 489S, T490K, 491T, 527K, E528D, G529D, 530E, D531E, R532K, S585R, A586G, Q587N, A588R) can be graphed onto an AAV9 capsid protein for and/or the AAV9 capsid protein can comprise Tyr mutations (AAV2 numbering: Y252F, Y272F, Y444F, Y500F, Y700F, Y704F, Y730F), galactose motif (AAV2 numbering: 469N, 470M, 471A, 472V, 474G, 500F) insertion of peptide with the amino acid abbreviation LALGETTRPA (AAV2 numbering: 587), or substitution mutation (AAV2 numbering: S265), in any combination. Amino acid residue numbering is based on the amino acid sequence of AAV2 (SEQ ID NO:2).

In some embodiments, the heparan sulfate binding motif (AAV2 numbering: 484R, 485Q, 486Q, 487R, 488V, 489S, T490K, 491T, 527 K, 528D, 529D, 530E, 531E, R532K, Q585R, A586G, 587N, T588R) can be graphed onto an AAV10 capsid protein and/or the AAV10 capsid protein can comprise Tyr mutations (AAV2 numbering: Y252F, Y272F, Y444F, Y500F, Y700F, Y704F, Y730F,), galactose motif (AAV2 numbering: 469N, 470M, S471A, A472V, A474G, 500F) insertion of peptide with the amino acid abbreviation LALGETTRPA (AAV2 numbering: 587), or substitution mutation (AAV2 numbering: S265), in any combination. Amino acid residue numbering is based on the amino acid sequence of AAV2 (SEQ ID NO:2).

In some embodiments, the heparan sulfate binding motif (AAV2 numbering: K484R, 485Q, 486Q, 487R, F488V, 489S, 490K, 491T, G527K, P528D, S529D, D530E, G531E, D532K, N585R, A586G, T587N, T588R) can be graphed onto an AAV11 capsid protein in any combination and/or the AAV11 capsid protein can comprise Tyr mutations (AAV2 numbering: Y252F, Y272F, Y444F, Y500F, Y700F, Y704F, Y730F), galactose motif (AAV2 numbering: D469N, F470M, 471A, F472V, R474G, A500F) insertion of peptide with the amino acid abbreviation LALGETTRPA (AAV2 numbering: 587). or insertion mutation (AAV2 numbering: 265), in any combination. Amino acid residue numbering is based on the amino acid sequence of AAV2 (SEQ ID NO:2).

In some embodiments, the heparan sulfate binding motif (AAV2 numbering: K484R, 485Q, 486Q, K487R, F488V, 489S, 490K, N491T, G527K, A528D, G529D, D530E, S531E, D532K, N585R, A586G, T587N, T588R) can be graphed onto an AAV12 capsid protein and/or the AAV12 capsid protein can comprise Tyr mutations (AAV2 numbering: Y252F, Y272F, Y444F, Y500F, Y700F, Y704F, Y730F), galactose motif (AAV2 numbering: D469N, F470M, 471A, F472V, R474G, A500F) insertion of peptide with the amino acid abbreviation LALGETTRPA (AAV2 numbering: 587), or insertion mutation (AAV2 numbering: 265), in any combination. Amino acid residue numbering is based on the amino acid sequence of AAV2 (SEQ ID NO:2).

In the methods of this invention, the viral vector can comprise a nucleic acid molecule that encodes a therapeutic protein and/or therapeutic DNA.

The present invention further provides a method of treating a disorder or defect of the eye in a subject, comprising the intravitreally administering viral vector of this invention to the subject receiving a therapeutic protein or therapeutic DNA effective in treating the disorder or defect of the eye in the subject.

Nonlimiting examples of a disorder or defect of the eye that can be treated according to the methods of this invention include age-related macular degeneration, Lebers congenital amarousis type 1, Lebers, congenital amarousis type 2, retinitis pigmentosa, retinoschosis, achromatopsia, color blindness, congenital stationary night blindness or any combination thereof.

### Definitions

The following terms are used in the description herein and the appended claims:

The singular forms “a,” “an” and “the” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” (and grammatical variants) means that the scope of a claim is to be interpreted to encompass the specified materials or steps recited in the claim, “and those that do not materially affect the basic and novel characteristic(s)” of the claimed invention. Thus, the term “consisting essentially of” when used in a claim of this invention is not intended to be interpreted to be equivalent to “comprising.”

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed. For example, in particular embodiments the amino acid is not A, G or I; is not A; is not G or V; etc. as if each such possible disclaimer is expressly set forth herein.

As used herein, the terms “reduce,” “reduces,” “reduction” and similar terms mean a decrease of at least about 25%, 35%, 50%, 75%, 80%, 85%, 90%, 95%, 97% or more.

As used herein, the terms “enhance,” “enhances,” “enhancement” and similar terms indicate an increase of at least about 5%, 10%, 20%, 25%, 50%, 75%, 100%, 150%, 200%, 300%, 400%, 500% or more. These terms can also be used in reference to fold increases, e.g., one-fold, two-fold, three-fold, four-fold, five-fold, six-fold, seven-fold, eight-fold, nine-fold, ten-fold, etc.

The term “parvovirus” as used herein encompasses the family Parvoviridae, including autonomously replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, volume 2, chapter 69 (4th ed., Lippincott-Raven Publishers).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of relatively new AAV serotypes and clades have been identified (see, e.g., Gao et al. (2004) J. Virology 78:6381-6388; Moris et al. (2004) Virology 33-:375-383; and Table 1).

The genomic sequences of various serotypes of AAV and the autonomous parvoviruses, as well as the sequences of the native terminal repeats (TRs), Rep proteins, and capsid subunits are known in the art. Such sequences may be found in the literature or in public databases such as the GenBank® Database. See, e.g., GenBank Accession Numbers NC_044927, NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, NC_001358, NC_001540, AF513851, AF513852, AY530579; the disclosures of which are incorporated by reference herein for teaching parvovirus and AAV nucleic acid and amino acid sequences. See also, e.g., Srivistava et al. (1983) J. Virology 45:555; Chiorini et al. (1998) J. Virology 71:6823; Chiorini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99:11854; Moris et al. (2004) Virology 33-: 375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1.

The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al. (2002) Proc. Nat. Acad. Sci. 99:10405-10), AAV4 (Padron et al. (2005) J. Virol. 79:5047-58), AAV5 (Walters et al. (2004) J. Virol. 78:3361-71) and CPV (Xie et al. (1996) J. Mol. Biol. 6:497-520 and Tsao et al. (1991) Science 251:1456-64).

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest. Those skilled in the art will appreciate that transcription of a heterologous nucleic acid sequence from the viral genome may not be initiated in the absence of trans-acting factors, e.g., for an inducible promoter or otherwise regulated nucleic acid sequence. In the case of a rAAV genome, gene expression from the viral genome may be from a stably integrated provirus, from a non-integrated episome, as well as any other form the virus may take within the cell.

Unless indicated otherwise, “efficient transduction” or “efficient tropism,” or similar terms, can be determined by reference to a suitable control (e.g., at least about 50%, 60%, 70%, 80%, 85%, 90%, 95%, 98%, 99%, 100% or more of the transduction or tropism, respectively, of the control). Suitable controls will depend on a variety of factors including the desired tropism profile.

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotides), but in representative embodiments are either single or double stranded DNA sequences.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, 100-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

As used herein, by “isolate” or “purify” (or grammatical equivalents) a virus vector, it is meant that the virus vector is at least partially separated from at least some of the other components in the starting material. In representative embodiments an “isolated” or “purified” virus vector is enriched by at least about 10-fold, 100-fold, 1000-fold, 10,000-fold or more as compared with the starting material. A “therapeutic protein” is a protein that can alleviate, reduce, prevent, delay and/or stabilize symptoms that result from an absence or defect in a protein in a cell or subject and/or is a protein that otherwise confers a benefit to a subject. A “therapeutic RNA molecule” or “functional RNA molecule” as used herein can be an antisense nucleic acid, a ribozyme (e.g., as described in U.S. Pat. No. 5,877,022), an RNA that effects spliceosome-mediated trans-splicing (see, Puttaraju et al. (1999) Nature Biotech. 17:246; U.S. Pat. Nos. 6,013,487; 6,083,702), an interfering RNA (RNAi) including siRNA, shRNA or miRNA, which mediate gene silencing (see, Sharp et al., (2000) Science 287:2431), and any other non-translated RNA, such as a “guide” RNA (Gorman et al. (1998) Proc. Nat. Acad. Sci. USA 95:4929; U.S. Pat. No. 5,869,248 to Yuan et al.) and the like as are known in the art.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The terms “heterologous nucleotide sequence” and “heterologous nucleic acid molecule” are used interchangeably herein and refer to a nucleic acid molecule and/or nucleotide sequence that is not naturally occurring in the virus. Generally, the heterologous nucleic acid comprises an open reading frame that encodes a protein, protein fragment, peptide or nontranslated RNA of interest (e.g., for delivery to a cell or subject).

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

A “rAAV vector genome” or “rAAV genome” is an AAV genome (i.e., vDNA) that comprises one or more heterologous nucleic acid sequences. rAAV vectors generally require only the terminal repeat(s) (TR(s)) in cis to generate virus. All other viral sequences are dispensable and may be supplied in trans (Muzyczka (1992) Curr. Topics Microbiol. Immunol. 158:97). Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention, the rAAV vector genome comprises at least one terminal repeat (TR) sequence (e.g., AAV TR sequence), optionally two TRs (e.g., two AAV TRs), which typically will be at the 5′ and 3′ ends of the vector genome and flank the heterologous nucleic acid sequence, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR” may be from any AAV, including but not limited to serotypes 1, 2, 3, 4, 5, 6, 7, 8, 9, 10 or 11 or any other AAV now known or later discovered (see, e.g., Table 1). An AAV terminal repeat need not have the native terminal repeat sequence (e.g., a native AAV TR sequence may be altered by insertion, deletion, truncation and/or missense mutations), as long as the terminal repeat mediates the desired functions, e.g., replication, virus packaging, integration, and/or provirus rescue, and the like.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al. (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

Further, the viral capsid or genomic elements can contain other modifications, including insertions, deletions and/or substitutions.

As used herein, the term “amino acid” encompasses any naturally occurring amino acid, modified forms thereof, and synthetic amino acids.

Naturally occurring, levorotatory (L-) amino acids are shown in Table 2.

Alternatively, the amino acid can be a modified amino acid residue (nonlimiting examples are shown in Table 3) and/or can be an amino acid that is modified by post-translation modification (e.g., acetylation, amidation, formylation, hydroxylation, methylation, phosphorylation or sulfatation).

Further, the non-naturally occurring amino acid can be an “unnatural” amino acid as described by Wang et al. Annu Rev Biophys Biomol Struct. 35:225-49 (2006)). These unnatural amino acids can advantageously be used to chemically link molecules of interest to the AAV capsid protein.

### Modified AAV Capsid Proteins and Virus Capsids and Virus Vectors Comprising the Same

The present invention provides AAV capsid proteins comprising a mutation (i.e., a modification) in the amino acid sequence and virus capsids and virus vectors comprising the modified AAV capsid protein. The inventors have discovered that modifications such as substitutions at the amino acid positions described herein can confer one or more desirable properties to virus vectors comprising the modified AAV capsid protein including without limitation selective transduction of cells having heparin sulfate on the surface and enhanced transduction of cells of the retina and/or retinal pigment epithelium.

In particular embodiments, the modified AAV capsid protein of the invention comprises one or more mutations (e.g., substitutions) in the amino acid sequence of the native AAV4 capsid protein or the corresponding region of a capsid protein from another AAV, including but not limited to AAV5. AAV7, AAV8 and AAV9.

As used herein, a “mutation” or “modification” in an amino acid sequence includes substitutions, insertions and/or deletions, each of which can involve one, two, three, four, five, six, seven, eight, nine, ten or more amino acids. In particular embodiments, the modification is a substitution. For example, in particular embodiments, the AAV4 capsid protein sequence is modified at amino acid positions 530, 584, 585, 586 and/or 587, in any combination.

In certain embodiments as described herein, amino acid residue numbering is based on the amino acid sequence of an AAV4 capsid protein having GenBank Accession No. NP_044927 (SEQ ID NO:4):

MTDGYLPDWLEDNLSEGVREWWALQPGAPKPKANQQHQDNARGLVLPGYK
YLGPGNGLDKGEPVNAADAAALEHDKAYDQQLKAGDNPYLKYNHADAEFQ
QRLQGDTSFGGNLGRAVFQAKKRVLEPLGLVEQAGETAPGKKRPLIESPQ
QPDSSTGIGKKGKQPAKKKLVFEDETGAGDGPPEGSTSGAMSDDSEMRAA
AGGAAVEGGQGADGVGNASGDWHCDSTWSEGHVTTTSTRTWVLPTYNNHL
YKRLGESLQSNTYNGFSTPWGYFDFNRFHCHFSPRDWQRLINNNWGMRPK
AMRVKIFNIQVKEVTTSNGETTVANNLTSTVQIFADSSYELPYVMDAGQE
GSLPPFPNDVFMVPQYGYCGLVTGNTSQQQTDRNAFYCLEYFPSQMLRTG
NNFEITYSFEKVPFHSMYAHSQSLDRLMNPLIDQYLWGLQSTTTGTTLNA
GTATTNFTKLRPTNFSNFKKNWLPGPSIKQQGFSKTANQNYKIPATGSDS
LIKYETHSTLDGRWSALTPGPPMATAGPADSKFSNSQLIFAGPKQNGNTA
TVPGTLIFTSEEELAATNATDTDMWGNLPGGDQSNSNLPTVDRLTALGAV
PGMVWQNRDIYYQGPIWAKIPHTDGHFHPSPLIGGFGLKHPPPQIFIKNT
PVPANPATTFSSTPVNSFITQYSTGQVSVQIDWEIQKERSKRWNPEVQFT
SNYGQQNSLLWAPDAAGKYTEPRAIGTRYLTHHL.

In certain embodiments as described herein, amino acid residue numbering is based on the amino acid sequence of an AAV2 capsid protein having GenBank Accession No. YP_680426 (SEQ ID NO:2):

MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNRQAATADVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL

In certain embodiments as described herein, amino acid residue numbering is based on the amino acid sequence of an AAV9 capsid protein having GenBank Accession No. AAS99264 (SEQ ID NO:8):

MAADGYLPDLEDNLSEGIEWWALKPGAQPKANQQHQNARGLVLPGKYLGP
GNGLDKGEPVNAADAALEHDKAYQQLKAGDNPLKYNHADAEQERLKEDTS
GGNLGRAVFQAKKRLLEPLLVEEAAKTAGKKRPVEQSQEPDSSAGIKSGA
QPAKKLNFGQTGDTESVPDPQPIGPPAAPSGVGLTMASGGGAVADNNEGA
DVGSSSGNWHDSQWLGDRVITTSTRTWALTYNNHLYKQSNSTSGGSSDNA
YFGYSTWGYFDFNRFCHFSPRDWQRLINNNWGFRKRLNFKLFNQVKEVTD
NNVKTIANNLTTVQVFTDSDQLPYVLGSAHEGCLPPFPAVFMIPQYGYTL
NDGSQAVRSSFYCLEYPSQMLRTGNFQFSYEFENVPFHSSYAHSSLDRLM
NPLDQYLYYLSKINGSGQNQQLKFSVAGPSMAVQGRNYIPGPSYRQQRVT
TVTQNNNSFAWPGASSWLNGRNSLMNGPAMASHKEEDRFFPLSGSLIFGK
QGTGDNVDADKVMTNEEEIKTTPVATESYGQATNHQSAQAAQTGWVQNQG
ILPGMVWQDDVYLQGPIWKIPHTDGNFPSPLMGGFGKHPPPQILINTPVP
ADPPTAFNKDKLNSITQYSTGQVVEIEWELQKNSKRWNPEIYTSNYYKSN
VEFAVNTEGVYSEPRPIGTYLTRNL

In certain embodiments as described herein, amino acid residue numbering is based on the amino acid sequence of an AAV5 capsid protein having GenBank Accession No. YP_068409 (SEQ ID NO:5):

MSFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEFQ
EKLADDTSFGGNLGKAVFQAKKRVLEPFGLVEEGAKTAPTGKRIDDHFPK
RKKARTEEDSKPSTSSDAEAGPSGSQQLQIPAQPASSLGADTMSAGGGGP
LGDNNQGADGVGNASGDWHCDSTWMGDRVVTKSTRTWVLPSYNNHQYREI
KSGSVDGSNANAYFGYSTPWGYFDENRFHSHWSPRDWQRLINNYWGFRPR
SLRVKIFNIQVKEVTVQDSTTTIANNLTSTVQVFTDDDYQLPYVVGNGTE
GCLPAFPPQVFTLPQYGYATLNRDNTENPTERSSFFCLEYFPSKMLRTGN
NFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGA
SYQVPPQPNGMTNNLQGSNTYALENTMIFNSQPANPGTTATYLEGNMLIT
SESETQPVNRVAYNVGGQMATNNQSSTTAPATGTYNLQEIVPGSVWMERD
VYLQGPIWAKIPETGAHFHPSPAMGGEGLKHPPPMMLIKNTPVPGNITSF
SDVPVSSFITQYSTGQVTVEMEWELKKENSKRWNPEIQYTNNYNDPQFVD
FAPDSTGEYRTTRPIGTRYLTRPL

In certain embodiments as described herein, amino acid numbering is based on the amino acid sequence of an AAV1 capsid protein having GenBank Accession No. NP_049542 (SEQ ID NO:1):

MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEQSP
QEPDSSSGIGKTGQQPAKKRLNFGQTGDSESVPDPQPLGEPPATPAAVGP
TTMASGGGAPMADNNEGADGVGNASGNWHCDSTWLGDRVITTSTRTWALP
TYNNHLYKQISSASTGASNDNHYFGYSTPWGYFDFNRFHCHFSPRDWQRL
INNNWGFRPKRLNFKLFNIQVKEVTTNDGVTTIANNLTSTVQVFSDSEYQ
LPYVLGSAHQGCLPPFPADVFMIPQYGYLTLNNGSQAVGRSSFYCLEYFP
SQMLRTGNNFTFSYTFEEVPFHSSYAHSQSLDRLMNPLIDQYLYYLNRTQ
NQSGSAQNKDLLFSRGSPAGMSVQPKNWLPGPCYRQQRVSKTKTDNNNSN
FTWTGASKYNLNGRESIINPGTAMASHKDDEDKFFPMSGVMIFGKESAGA
SNTALDNVMITDEEEIKATNPVATERFGTVAVNFQSSSTDPATGDVHAMG
ALPGMVWQDRDVYLQGPIWAKIPHTDGHFHPSPLMGGFGLKNPPPQILIK
NTPVPANPPAEFSATKFASFITQYSTGQVSVEIEWELQKENSKRWNPEVQ
YTSNYAKSANVDFTVDNNGLYTEPRPIGTRYLTRPL

In certain embodiments as described herein, amino acid residue numbering is based on the amino acid sequence of an AAV7 capsid protein having GenBank Accession No. YP_077178 (SEQ ID NO:6):

MAADGYLPDWLEDNLSEGIREWWDLKPGAPKPKANQQKQDNGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPAKKRPVEPSP
QRSPDSSTGIGKKGQQPARKRLNFGQTGDSESVPDPQPLGEPPAAPSSVG
SGTVAAGGGAPMADNNEGADGVGNASGNWHCDSTWLGDRVITTSTRTWAL
PTYNNHLYKQISSETAGSTNDNTYFGYSTPWGYFDFNRFHCHFSPRDWQR
LINNNWGFRPKKLRFKLFNIQVKEVTTNDGVTTIANNLTSTIQVFSDSEY
QLPYVLGSAHQGCLPPFPADVFMIPQYGYLTLNNGSQSVGRSSFYCLEYF
PSQMLRTGNNFEFSYSFEDVPFHSSYAHSQSLDRLMNPLIDQYLYYLART
QSNPGGTAGNRELQFYQGGPSTMAEQAKNWLPGPCFRQQRVSKTLDQNNN
SNFAWTGATKYHLNGRNSLVNPGVAMATHKDDEDRFFPSSGVLIFGKTGA
TNKTTLENVLMTNEEEIRPTNPVATEEYGIVSSNLQAANTAAQTQVVNNQ
GALPGMVWQNRDVYLQGPIWAKIPHTDGNFHPSPLMGGFGLKHPPPQILI
KNTPVPANPPEVFTPAKFASFITQYSTGQVSVEIEWELQKENSKRWNPEI
QYTSNFEKQTGVDFAVDSQGVYSEPRPIGTRYLTRNL

In certain embodiments as described herein, amino acid residue numbering is based on the amino acid sequence of an AAV8 capsid protein having GenBank Accession No. YP_077180 (SEQ ID NO:7):

MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGLVEEGAKTAPGKKRPVEPSP
QRSPDSSTGIGKKGQQPARKRLNFGQTGDSESVPDPQPLGEPPAAPSGVG
PNTMAAGGGAPMADNNEGADGVGSSSGNWHCDSTWLGDRVITTSTRTWAL
PTYNNHLYKQISNGTSGGATNDNTYFGYSTPWGYFDFNRFHCHFSPRDWQ
RLINNNWGFRPKRLSFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFQFTYTFEDVPFHSSYAHSQSLDRLMNPLIDQYLYYLSR
TQTTGGTANTQTLGFSQGGPNTMANQAKNWLPGPCYRQQRVSTTTGQNNN
SNFAWTAGTKYHLNGRNSLANPGIAMATHKDDEERFFPSNGILIFGKQNA
ARDNADYSDVMLTSEEEIKTTNPVATEEYGIVADNLQQQNTAPQIGTVNS
QGALPGMVWQNRDVYLQGPIWAKIPHTDGNFHPSPLMGGFGLKHPPPQIL
IKNTPVPADPPTTFNQSKLNSFITQYSTGQVSVEIEWELQKENSKRWNPE
IQYTSNYYKSTSVDFAVNTEGVYSEPRPIGTRYLTRNL.

In certain embodiments as described herein, amino acid residue numbering is based on the amino acid sequence of an AAV10 capsid protein having GenBank Accession No.

AAT46337 (SEQ ID NO:9):

MAADGYLPDWLEDNLSEGIREWWDLKPGAPKPKANQQKQDDGRGLVLPGY
KYLGPFNGLD61KGEPVNAADAAALEHDKAYDQQLKAGDNPYLRYNHADA
EFQERLQEDTSFGGNLGRAVFQ121AKKRVLEPLGLVEEAAKTAPGKKRP
VEPSPQRSPDSSTGIGKKGQQPAKKRLNFGQTGES181ESVPDPQPIGEP
PAGPSGLGSGTMAAGGGAPMADNNEGADGVGSSSGNWHCDSTWLGDRV
241ITTSTRTWALPTYNNHLYKQISNGTSGGSTNDNTYFGYSTPWGYFDF
NRFHCHFSPRDWQ301RLINNNWGFRPKRLSFKLFNIQVKEVTQNEGTKT
IANNLTSTIQVFTDSEYQLPYVLGSA361HQGCLPPFPADVFMIPQYGYL
TLNNGSQAVGRSSFYCLEYFPSQMLRTGNNFEFSYTFED421VPFHSSYA
HSQSLDRLMNPLIDQYLYYLSRTQSTGGTQGTQQLLFSQAGPANMSAQAK
NW481LPGPCYRQQRVSTTLSQNNNSNFAWTGATKYHLNGRDSLVNPGVA
MATHKDDEERFFPSS541GVLMFGKQGAGRDNVDYSSVMLTSEEEIKTTN
PVATEQYGVVADNLQQANTGPIVGNVNS601QGALPGMVWQNRDVYLQGP
IWAKIPHTDGNFHPSPLMGGFGLICHPPPQILIKNTPVPADP661PTTFS
QAKLASFITQYSTGQVSVEIEWELQKENSKRWNPEIQYTSNYYKSTNVDF
AVNTE721GTYSEPRPIGTRYLTRNL.

In certain embodiments as described herein, amino acid residue numbering is based on the amino acid sequence of an AAV11 capsid protein having GenBank Accession No. AAT46339 (SEQ ID NO:10):

MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPLESPQ
EPDSSSGIGKKGKQPARKRLNFEEDTGAGDGPPEGSDTSAMSSDIEMRAA
PGGNAVDAGQGSDGVGNASGDWHCDSTWSEGKVTTTSTRTWVLPTYNNHL
YLRLGTTSSSNTYNGFSTPWGYFDFNRFHCHFSPRDWQRLINNNWGLRPK
AMRVKIFNIQVKEVTTSNGETTVANNLTSTVQIFADSSYELPYVMDAGQE
GSLPPFPNDVFMVPQYGYCGIVTGENQNQTDRNAFYCLEYFPSQMLRTGN
NFEMAYNFEKVPFHSMYAHSQSLDRLMNPLLDQYLWHLQSTTSGETLNQG
NAATTFGKIRSGDFAFYRKNWLPGPCVKQQRFSKTASQNYKIPASGGNAL
LKYDTHYTLNNRWSNIAPGPPMATAGPSDGDFSNAQLIFPGPSVTGNTTT
SANNLLFTSEEEIAATNPRDTDMFGQIADNNQNATTAPITGNVTAMGVLP
GMVWQNRDIYYQGPIWAKIPHADGHFHPSPLIGGFGLKHPPPQIFIKNTP
VPANPATTFTAARVDSFITQYSTGQVAVQIEWEIEKERSKRWNPEVQFTS
NYGNQSSMLWAPDTTGKYTEPRVIGSRYLTNHL

In certain embodiments as described herein, amino acid residue numbering is based on the amino acid sequence of an AAV3B capsid protein having GenBank Accession No.

NC_001863 (SEQ ID NO:3):

MAADGYLPDWLEDNLSEGIREWWALKPGVPQPKANQQHQDNRRGLVLPGY
KYLGPGNGLDKGEPVNEADAAALEHDKAYDQQLKAGDNPYLKYNHADAEF
QERLQEDTSFGGNLGRAVFQAKKRILEPLGLVEEAAKTAPGKKRPVDQSP
QEPDSSSGVGKSGKQPARKRLNFGQTGDSESVPDPQPLGEPPAAPTSLGS
NTMASGGGAPMADNNEGADGVGNSSGNWHCDSQWLGDRVITTSTRTWALP
TYNNHLYKQISSQSGASNDNHYFGYSTPWGYFDFNRFHCHFSPRDWQRLI
NNNWGFRPKKLSFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNRTQG
TTSGTTNQSRLLFSQAGPQSMSLQARNWLPGPCYRQQRLSKTANDNNNSN
FPWTAASKYHLNGRDSLVNPGPAMASHKDDEEKFFPMHGNLIFGKEGTTA
SNAELDNVMITDEEEIRTTNPVATEQYGTVANNLQSSNTAPTTRTVNDQG
ALPGMVWQDRDVYLQGPIWAKIPHTDGHFHPSPLMGGFGLKHPPPQIMIK
NTPVPANPPTTFSPAKFASFITQYSTGQVSVEIEWELQKENSKRWNPEIQ
YTSNYNKSVNVDFTVDTNGVYSEPRPIGTRYLTRNL.

In certain embodiments as described herein, amino acid residue numbering is based on the amino acid sequence of an AAV12 capsid protein having GenBank Accession No. ABI16639 (SEQ ID NO:11):

MAADGYLPDW LEDNLSEGIR EWWALKPGAP QPKANQQHQD
NGRGLVLPGY KYLGPFNGLD KGEPVNEADA AALEHDKAYD
KQLEQGDNPY LKYNHADAEF QQRLATDTSF GGNLGRAVFQ
AKKRILEPLG LVEEGVKTAP GKKRPLEKTP NRPTNPDSGK
APAKKKQKDG EPADSARRTL DFEDSGAGDG PPEGSSSGEM
SHDAEMRAAP GGNAVEAGQG ADGVGNASGD WHCDSTWSEG
RVTTTSTRTW VLPTYNNHLY LRIGTTANSN TYNGFSTPWG
YFDFNRFHCH FSPRDWQRLI NNNWGLRPKS MRVKIFNIQV
KEVTTSNGET TVANNLTSTV QIFADSTYEL PYVMDAGQEG
SFPPFPNDVF MVPQYGYCGV VTGKNQNQTD RNAFYCLEYF
PSQMLRTGNN FEVSYQFEKV PFHSMYAHSQ SLDRMMNPLL
DQYLWHLQST TTGNSLNQGT ATTTYGKITT GDFAYYRKNW
LPGACIKQQK FSKNANQNYK IPASGGDALL KYDTHTTLNG
RWSNMAPGPP MATAGAGDSD FSNSQLIFAG PNPSGNTTTS
SNNLLFTSEE EIATTNPRDT DMFGQIADNN QNATTAPHIA
NLDAMGIVPG MVWQNRDIYY QGPIWAKVPH TDGHFHPSPL
MGGFGLKHPP PQIFIKNTPV PANPNTTFSA ARINSFLTQY
STGQVAVQID WEIQKEHSKR WNPEVQFTSN YGTQNSMLWA
PDNAGNYHEL RAIGSRFLTH HL.

The modified virus capsid proteins of the invention can be but are not limited to AAV capsid proteins in which amino acids from one AAV capsid protein are substituted into another AAV capsid protein, and the substituted and/or inserted amino acids can be from any source, and can further be naturally occurring or partially or completely synthetic. Furthermore, the AAV capsid proteins of this invention can have a native amino acid sequence or a synthetic amino acid sequence.

As described herein, the nucleic acid and amino acid sequences of the capsid proteins from a number of AAVs are known in the art. Thus, for example, the amino acid(s) “corresponding” to amino acid positions 530, 584, 585, 586 and 587 of the reference AAV4 capsid protein can be readily determined for any other AAV capsid protein, including, for example, AAV5, AAV7, AAV8 and AAV9 (e.g., by using sequence alignments as are well known in the art). The amino acid positions in other AAV serotypes or modified AAV capsids that “correspond to” these positions in the native AAV4 capsid protein will be apparent to those skilled in the art and can be readily determined using sequence alignment techniques (see, e.g., FIG. 7 of WO 2006/066066) and/or crystal structure analysis (Padron et al. (2005) J. Virol. 79:5047-58). Examples of amino acid residues that can be substituted for the native amino acid at these respective positions in other AAV serotype are set forth in Tables 2 and 3.

The invention contemplates that the modified capsid proteins of the invention can be produced by modifying the capsid protein of any AAV now known or later discovered. Further, the AAV capsid protein that is to be modified can be a naturally occurring AAV capsid protein (e.g., an AAV4, AAV5, AAV7, AAV8, or AAV9 capsid protein or any of the AAV shown in Table 1) but is not so limited. Those skilled in the art will understand that a variety of manipulations to the AAV capsid proteins are known in the art and the invention is not limited to modifications of naturally occurring AAV capsid proteins. For example, the capsid protein to be modified may already have alterations as compared with naturally occurring AAV (e.g., is derived from a naturally occurring AAV capsid protein, e.g., AAV1, AAV2, AAV3a, AAV3b, AAV4, AAV5, AAV6, AAV7, AAV8, AAV9, AAV10, AAV11 and/or AAV12 or any other AAV serotype now known or later discovered). Such AAV capsid proteins are also within the scope of the present invention.

Thus, in particular embodiments, the AAV capsid protein to be modified can be derived from a naturally occurring AAV but further comprise one or more foreign sequences (e.g., that are exogenous to the native virus) that are inserted and/or substituted into the capsid protein and/or has been altered by deletion of one or more amino acids.

Accordingly, when referring herein to a specific AAV capsid protein (e.g., an AAV1, AAV2, AAV3, AAV4, AAV5, AAV6, AAV7, AAV8, AAV9, AAV10, AAV11 or AAV12 capsid protein or a capsid protein from any of the AAV shown in Table 1, etc.), it is intended to encompass the native capsid protein as well as capsid proteins that have alterations other than the modifications of the invention. Such alterations include substitutions, insertions and/or deletions. In particular embodiments, the capsid protein comprises 1, 2, 3, 4, 5, 6, 7, 8, 9, 10, 11, 12, 13, 14, 15, 16, 17, 18, 19 or 20, less than 20, less than 30, less than 40 less than 50, less than 60, or less than 70 amino acids inserted therein (other than the insertions of the present invention) as compared with the native AAV capsid protein sequence. In embodiments of the invention, the capsid protein comprises 1, 2, 3, 4, 5, 6, 7, 8, 9, 10, 11, 12, 13, 14, 15, 16, 17, 18, 19 or 20, less than 20, less than 30, less than 40 less than 50, less than 60, or less than 70 amino acid substitutions (other than the amino acid substitutions according to the present invention) as compared with the native AAV capsid protein sequence. In embodiments of the invention, the capsid protein comprises a deletion of 1, 2, 3, 4, 5, 6, 7, 8, 9, 10, 11, 12, 13, 14, 15, 16, 17, 18, 19 or 20, less than 20, less than 30, less than 40 less than 50, less than 60, or less than 70 amino acids (other than the amino acid deletions of the invention) as compared with the native AAV capsid protein sequence.

Thus, for example, the term “AAV4 capsid protein” includes AAV capsid proteins having the native AAV4 capsid protein sequence (see GenBank Accession No. NC_044927) as well as those comprising substitutions, insertions and/or deletions (as described herein) in the native AAV4 capsid protein sequence.

In particular embodiments, the AAV capsid protein has the native AAV capsid protein sequence or has an amino acid sequence that is at least about 90%, 95%, 97%, 98% or 99% similar or identical to a native AAV capsid protein sequence. For example, in particular embodiments, an “AAV4” capsid protein encompasses the native AAV4 capsid protein sequence as well as sequences that are at least about 90%, 95%, 97%, 98% or 99% similar or identical to the native AAV4 capsid protein sequence.

Methods of determining sequence similarity or identity between two or more amino acid sequences are known in the art. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al. Nucl. Acid Res. 12, 387-395 (1984), or by inspection.

Another suitable algorithm is the BLAST algorithm, described in Altschul et al. J. Mol. Biol. 215, 403-410, (1990) and Karlin et al. Proc. Natl. Acad. Sci. USA 90, 5873-5787 (1993). A particularly useful BLAST program is the WU-BLAST-2 program which was obtained from Altschul et al. Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

Further, an additional useful algorithm is gapped BLAST as reported by Altschul et al., (1997) Nucleic Acids Res. 25, 3389-3402.

The modified virus capsids can be used as “capsid vehicles,” as has been described, for example, in U.S. Pat. No. 5,863,541. Molecules that can be packaged by the modified virus capsid and transferred into a cell include heterologous DNA, RNA, polypeptides, small organic molecules, metals, or combinations of the same.

Heterologous molecules are defined as those that are not naturally found in an AAV infection, e.g., those not encoded by a wild-type AAV genome. Further, therapeutically useful molecules can be associated with the outside of the chimeric virus capsid for transfer of the molecules into host target cells. Such associated molecules can include DNA, RNA, small organic molecules, metals, carbohydrates, lipids and/or polypeptides. In one embodiment of the invention the therapeutically useful molecule is covalently linked (i.e., conjugated or chemically coupled) to the capsid proteins. Methods of covalently linking molecules are known by those skilled in the art.

In other embodiments, the virus capsids can be administered to block certain cellular sites prior to and/or concurrently with (e.g., within minutes or hours of each other) administration of a virus vector delivering a nucleic acid encoding a polypeptide or functional RNA of interest. For example, the inventive capsids can be delivered to block cellular receptors on particular cells and a delivery vector can be administered subsequently or concurrently, which may reduce transduction of the blocked cells, and enhance transduction of other targets (e.g., CNS progenitor cells and/or neuroblasts).

According to representative embodiments, modified virus capsids can be administered to a subject prior to and/or concurrently with a modified virus vector according to the present invention. Further, the invention provides compositions and pharmaceutical formulations comprising the inventive modified virus capsids; optionally, the composition also comprises a modified virus vector of the invention.

Further provided herein is an AAV capsid comprising the AAV capsid protein described herein as well as a virus vector comprising the AAV capsid. Also provided herein is a composition comprising the virus vector of this invention in a pharmaceutically acceptable carrier.

The invention also provides nucleic acid molecules (optionally, isolated nucleic acid molecules) encoding the modified virus capsids and capsid proteins of the invention. Further provided are vectors comprising the nucleic acid molecules and cells (in vivo or in culture) comprising the nucleic acid molecules and/or vectors of the invention. Suitable vectors include without limitation viral vectors (e.g., adenovirus, AAV, herpesvirus, vaccinia, poxviruses, baculoviruses, and the like), plasmids, phage, YACs, BACs, and the like. Such nucleic acid molecules, vectors and cells can be used, for example, as reagents (e.g., helper packaging constructs or packaging cells) for the production of modified virus capsids or virus vectors as described herein.

Virus capsids according to the invention can be produced using any method known in the art, e.g., by expression from a baculovirus (Brown et al. (1994) Virology 198:477-488).

The modifications to the AAV capsid protein according to the present invention are “selective” modifications. This approach is in contrast to previous work with whole subunit or large domain swaps between AAV serotypes (see, e.g., international patent publication WO 00/28004 and Hauck et al. (2003) J. Virology 77:2768-2774). In particular embodiments, a “selective” modification results in the insertion and/or substitution and/or deletion of less than about 20, 18, 15, 12, 10, 9, 8, 7, 6, 5, 4, 3, 2 or 1 contiguous amino acids.

The modified capsid proteins and capsids of the invention can further comprise any other modification, now known or later identified.

For example, the AAV capsid proteins and virus capsids of the invention can be chimeric in that they can comprise all or a portion of a capsid subunit from another virus, optionally another parvovirus or AAV, e.g., as described in international patent publication WO 00/28004.

The virus capsid can be a targeted virus capsid comprising a targeting sequence (e.g., substituted or inserted in the viral capsid) that directs the virus capsid to interact with cell-surface molecules present on a desired target tissue(s) (see, e.g., international patent publication WO 00/28004 and Hauck et al. (2003) J. Virology 77:2768-2774); Shi et al. Human Gene Therapy 17:353-361 (2006) [describing insertion of the integrin receptor binding motif RGD at positions 520 and/or 584 of the AAV capsid subunit]; and U.S. Pat. No. 7,314,912 [describing insertion of the P1 peptide containing an RGD motif following amino acid positions 447, 534, 573 and 587 of the AAV2 capsid subunit]). Other positions within the AAV capsid subunit that tolerate insertions are known in the art (e.g., positions 449 and 588 described by Grifman et al. Molecular Therapy 3:964-975 (2001)).

In representative embodiments, the targeting sequence may be a virus capsid sequence (e.g., an autonomous parvovirus capsid sequence, AAV capsid sequence, or any other viral capsid sequence) that directs infection to a particular cell type(s).

As another nonlimiting example, a heparin binding domain (e.g., the respiratory syncytial virus heparin binding domain) may be inserted or substituted into a capsid subunit that does not typically bind HS receptors (e.g., AAV 4, AAV5) to confer heparin binding to the resulting mutant.

In representative embodiments, the exogenous targeting sequence may be any amino acid sequence encoding a peptide that alters the tropism of a virus capsid or virus vector comprising the modified AAV capsid protein. In particular embodiments, the targeting peptide or protein may be naturally occurring or, alternately, completely or partially synthetic. Exemplary targeting sequences include ligands and other peptides that bind to cell surface receptors and glycoproteins, such as RGD peptide sequences, bradykinin, hormones, peptide growth factors (e.g., epidermal growth factor, nerve growth factor, fibroblast growth factor, platelet-derived growth factor, insulin-like growth factors I and II, etc.), cytokines, melanocyte stimulating hormone (e.g., α, β or γ), neuropeptides and endorphins, and the like, and fragments thereof that retain the ability to target cells to their cognate receptors. Other illustrative peptides and proteins include substance P, keratinocyte growth factor, neuropeptide Y, gastrin releasing peptide, interleukin 2, hen egg white lysozyme, erythropoietin, gonadoliberin, corticostatin, β-endorphin, leu-enkephalin, rimorphin, α-neo-enkephalin, angiotensin, pneumadin, vasoactive intestinal peptide, neurotensin, motilin, and fragments thereof as described above. As yet a further alternative, the binding domain from a toxin (e.g., tetanus toxin or snake toxins, such as α-bungarotoxin, and the like) can be substituted into the capsid protein as a targeting sequence. In a yet further representative embodiment, the AAV capsid protein can be modified by substitution of a “nonclassical” import/export signal peptide (e.g., fibroblast growth factor-1 and -2, interleukin 1, HIV-1 Tat protein, herpes virus VP22 protein, and the like) as described by Cleves (Current Biology 7:R318 (1997)) into the AAV capsid protein. Also encompassed are peptide motifs that direct uptake by specific cells, e.g., a FVFLP peptide motif triggers uptake by liver cells.

Phage display techniques, as well as other techniques known in the art, may be used to identify peptides that recognize any cell type of interest.

The targeting sequence may encode any peptide that targets to a cell surface binding site, including receptors (e.g., protein, carbohydrate, glycoprotein or proteoglycan). Examples of cell surface binding sites include, but are not limited to, heparan sulfate, chondroitin sulfate, and other glycosaminoglycans, sialic acid moieties, polysialic acid moieties, glycoproteins, and gangliosides, MHC I glycoproteins, carbohydrate components found on membrane glycoproteins, including, mannose, N-acetyl-galactosamine, N-acetyl-glucosamine, fucose, galactose, and the like.

As yet a further alternative, the targeting sequence may be a peptide that can be used for chemical coupling (e.g., can comprise arginine and/or lysine residues that can be chemically coupled through their R groups) to another molecule that targets entry into a cell.

The foregoing embodiments of the invention can be used to deliver a heterologous nucleic acid to a cell or subject as described herein. Thus, in one embodiment, the present invention provides a method of introducing a nucleic acid molecule into a cell, comprising contacting the cell with the virus vector and/or composition of this invention.

Further provided herein is a method of delivering a nucleic acid molecule to a subject, comprising administering to the subject the virus vector of this invention and/or the composition of this invention. In some embodiments, the virus vector or composition is administered to the central nervous system of the subject.

Additionally provided herein is a method of selectively transducing a cell having heparan sulfate on the surface, comprising contacting the cell with the virus vector of this invention and/or the composition of this invention.

The present invention further provides a method of delivering a nucleic acid molecule of interest to a cell of a retina and/or retinal pigment epithelium, comprising contacting the cell with the virus vector of this invention, wherein the virus vector comprises the nucleic acid molecule of interest. In some embodiments of this method, the nucleic acid molecule of interest encodes a therapeutic protein or therapeutic RNA.

In some embodiments of the methods described above, the cell of a retina and/or retinal pigment epithelium can be in a subject and in some embodiments, the subject can be a human subject.

The present invention further provides a method of treating a disorder or defect in the eye of a subject, comprising intravitreally administering to the subject the virus vector of this invention, wherein the virus vector comprises a nucleic acid molecule that encodes a therapeutic protein or therapeutic RNA effective in treating the disorder or defect in the eye of the subject.

In further embodiments, the present invention provides a method of selectively transducing a cell of a retina and/or retinal pigment epithelium, comprising contacting the cell with a virus vector comprising an AAV capsid protein as described herein.

Those skilled in the art will appreciate that for some AAV capsid proteins the corresponding modification will be an insertion and/or a substitution, depending on whether the corresponding amino acid positions are partially or completely present in the virus or, alternatively, are completely absent. Likewise, when modifying AAV other than AAV4, the specific amino acid position(s) may be different than the position in AAV4 (see, e.g., Table 4, which shows a representative example of amino acid residues corresponding to S257 in AAV4). As discussed elsewhere herein, the corresponding amino acid position(s) will be readily apparent to those skilled in the art using well-known techniques.

The modifications described above can be incorporated into the capsid proteins or capsids of the invention in combination with each other and/or with any other modification now known or later discovered.

The invention also encompasses virus vectors comprising the modified capsid proteins and capsids of the invention. In particular embodiments, the virus vector is a parvovirus vector (e.g., comprising a parvovirus capsid and/or vector genome), for example, an AAV vector (e.g., comprising an AAV capsid and/or vector genome). In representative embodiments, the virus vector comprises a modified AAV capsid comprising a modified capsid subunit of the invention and a vector genome.

For example, in representative embodiments, the virus vector comprises: (a) a modified virus capsid (e.g., a modified AAV capsid) comprising a modified capsid protein of the invention; and (b) a nucleic acid comprising a terminal repeat sequence (e.g., an AAV TR), wherein the nucleic acid comprising the terminal repeat sequence is encapsidated by the modified virus capsid. The nucleic acid can optionally comprise two terminal repeats (e.g., two AAV TRs).

In representative embodiments, the virus vector is a recombinant virus vector comprising a heterologous nucleic acid molecule encoding a protein, peptide and/or functional RNA of interest.

It will be understood by those skilled in the art that the modified capsid proteins, virus capsids and virus vectors of the invention exclude those capsid proteins, capsids and virus vectors that have the indicated amino acids at the specified positions in their native state (i.e., are not mutants).

### Recombinant Virus Vectors

The virus vectors of the present invention are useful for the delivery of nucleic acids to cells in vitro, ex vivo, and in vivo. In particular, the virus vectors can be advantageously employed to deliver or transfer nucleic acids to animal, including mammalian, cells.

Any heterologous nucleic acid sequence(s) of interest may be delivered in the virus vectors of the present invention. Nucleic acids of interest include nucleic acids encoding polypeptides, including therapeutic (e.g., for medical or veterinary uses) or immunogenic (e.g., for vaccines) proteins and/or functional or therapeutic RNA molecules.

Heterologous nucleic acid sequences encoding polypeptides include those encoding reporter polypeptides (e.g., an enzyme). Reporter polypeptides are known in the art and include, but are not limited to, green fluorescent protein (GFP), β-galactosidase, alkaline phosphatase, luciferase, and chloramphenicol acetyltransferase gene.

Optionally, the heterologous nucleic acid encodes a secreted polypeptide (e.g., a polypeptide that is a secreted polypeptide in its native state or that has been engineered to be secreted, for example, by operable association with a secretory signal sequence as is known in the art).

Alternatively, in particular embodiments of this invention, the heterologous nucleic acid may encode an antisense nucleic acid, a ribozyme (e.g., as described in U.S. Patent No. 5,877,022), RNAs that effect spliceosome-mediated trans-splicing (see, Puttaraju et al. (1999) Nature Biotech. 17:246; U.S. Pat. Nos. 6,013,487; 6,083,702), interfering RNAs (RNAi) including siRNA, shRNA or miRNA that mediate gene silencing (see, Sharp et al. (2000) Science 287:2431), and other non-translated RNAs, such as “guide” RNAs (Gorman et al. (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al. J. Gene Med. 10:132-142 (2008) and Li et al. Acta Pharmacol Sin. 26:51-55 (2005)); phospholamban inhibitory or dominant-negative molecules such as phospholamban S16E (e.g., to treat cardiovascular disease, see, e.g., Hoshijima et al. Nat. Med. 8:864-871 (2002)), RNAi to adenosine kinase (e.g., for epilepsy), and RNAi directed against pathogenic organisms and viruses (e.g., hepatitis B and/or C virus, human immunodeficiency virus, CMV, herpes simplex virus, human papilloma virus, etc.).

Further, a nucleic acid sequence that directs alternative splicing can be delivered. To illustrate, an antisense sequence (or other inhibitory sequence) complementary to the 5′ and/or 3′ splice site of dystrophin exon 51 can be delivered in conjunction with a U1 or U7 small nuclear (sn) RNA promoter to induce skipping of this exon. For example, a DNA sequence comprising a U1 or U7 snRNA promoter located 5′ to the antisense/inhibitory sequence(s) can be packaged and delivered in a modified capsid of the invention.

The virus vector may also comprise a heterologous nucleic acid that shares homology with and recombines with a locus on a host chromosome. This approach can be utilized, for example, to correct a genetic defect in the host cell.

As a further alternative, the heterologous nucleic acid can encode any polypeptide that is desirably produced in a cell in vitro, ex vivo, or in vivo. For example, the virus vectors may be introduced into cultured cells and the expressed gene product isolated therefrom.

It will be understood by those skilled in the art that the heterologous nucleic acid(s) of interest can be operably associated with appropriate control sequences. For example, the heterologous nucleic acid can be operably associated with expression control elements, such as transcription/translation control signals, origins of replication, polyadenylation signals, internal ribosome entry sites (IRES), promoters, and/or enhancers, and the like.

Further, regulated expression of the heterologous nucleic acid(s) of interest can be achieved at the post-transcriptional level, e.g., by regulating selective splicing of different introns by the presence or absence of an oligonucleotide, small molecule and/or other compound that selectively blocks splicing activity at specific sites (e.g., as described in WO 2006/119137).

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the heterologous nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the heterologous nucleic acid sequence(s). Inducible promoters/enhancer elements for gene delivery can be tissue-specific or—preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the heterologous nucleic acid sequence(s) is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

The virus vectors according to the present invention provide a means for delivering heterologous nucleic acids into a broad range of cells, including dividing and non-dividing cells. The virus vectors can be employed to deliver a nucleic acid of interest to a cell in vitro, e.g., to produce a polypeptide in vitro or for ex vivo gene therapy. The virus vectors are additionally useful in a method of delivering a nucleic acid to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The virus vectors can also be used to produce a polypeptide of interest or functional RNA in cultured cells or in a subject (e.g., using the subject as a bioreactor to produce the polypeptide or to observe the effects of the functional RNA on the subject, for example, in connection with screening methods).

In general, the virus vectors of the present invention can be employed to deliver a heterologous nucleic acid encoding a polypeptide or functional RNA to treat and/or prevent any disease state for which it is beneficial to deliver a therapeutic polypeptide or functional RNA. Illustrative disease states of this invention include, but are not limited to, age-related macular degeneration, Lebers congenital amarousis type 1, Lebers, congenital amarousis type 2, retinitis pigmentosa, retinoschosis, achromatopsia, color blindness, congenital stationary night blindness or any combination thereof.

Gene transfer has substantial potential use for understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, virus vectors according to the present invention permit the treatment and/or prevention of genetic diseases.

The virus vectors according to the present invention may also be employed to provide a functional RNA to a cell in vitro or in vivo. Expression of the functional RNA in the cell, for example, can diminish expression of a particular target protein by the cell. Accordingly, functional RNA can be administered to decrease expression of a particular protein in a subject in need thereof. Functional RNA can also be administered to cells in vitro to regulate gene expression and/or cell physiology, e.g., to optimize cell or tissue culture systems or in screening methods.

In addition, virus vectors according to the instant invention find use in diagnostic and screening methods, whereby a nucleic acid of interest is transiently or stably expressed in a cell culture system, or alternatively, a transgenic animal model.

In some embodiments, the virus vectors of the present invention can be used to induce an immune response in a subject and the virus vector can comprise a nucleotide sequence that encodes an immunogen.

The virus vectors of the present invention can also be used for various non-therapeutic purposes, including but not limited to use in protocols to assess gene targeting, clearance, transcription, translation, etc., as would be apparent to one skilled in the art. The virus vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

Alternatively, the virus vector may be administered to a cell ex vivo and the altered cell is administered to the subject. The virus vector comprising the heterologous nucleic acid is introduced into the cell, and the cell is administered to the subject, where the heterologous nucleic acid can be expressed in the subject.

### Subjects, Pharmaceutical Formulations, and Modes of Administration

Virus vectors and capsids according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, rodents, bovines, ovines, caprines, equines, felines, canines, lagomorphs, etc. Human subjects include neonates, infants, juveniles, adults and geriatric subjects.

In representative embodiments, the subject is “in need of” the methods of the invention.

In particular embodiments, the present invention provides a pharmaceutical composition comprising a virus vector and/or capsid of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

One aspect of the present invention is a method of transferring a nucleic acid to a cell in vitro. The virus vector may be introduced into the cells at the appropriate multiplicity of infection according to standard transduction methods suitable for the particular target cells. Titers of virus vector to administer can vary, depending upon the target cell type and number, and the particular virus vector, and can be determined by those of skill in the art without undue experimentation. In representative embodiments, at least about 10³ infectious units, optionally at least about 10⁵ infectious units are introduced to the cell.

The cell(s) into which the virus vector is introduced can be of any type, including but not limited to cells of the eye (including retinal cells, retinal pigment epithelium, and corneal cells).

The virus vector can be introduced into cells in vitro for the purpose of administering the modified cell to a subject. In particular embodiments, the cells have been removed from a subject, the virus vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the recombinant virus vector can be introduced into cells from a donor subject, into cultured cells, or into cells from any other suitable source, and the cells are administered to a subject in need thereof (i.e., a “recipient” subject).

Suitable cells for ex vivo nucleic acid delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the virus vector are administered to the subject in a treatment effective or prevention effective amount in combination with a pharmaceutical carrier.

A further aspect of the invention is a method of administering the virus vector and/or virus capsid to a subject. Administration of the virus vectors and/or capsids according to the present invention to a human subject or an animal in need thereof can be by any means known in the art, but in particular embodiments, administration is intravitreal. Optionally, the virus vector and/or capsid is delivered in a treatment effective or prevention effective dose in a pharmaceutically acceptable carrier.

Dosages of the virus vector and/or capsid to be administered to a subject depend upon the mode of administration, the disease or condition to be treated and/or prevented, the individual subject's condition, the particular virus vector or capsid, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10³, 10¹⁴, 10¹⁵ transducing units, optionally about 10⁸-10¹³ transducing units.

In particular embodiments, more than one administration (e.g., two, three, four or more administrations) may be employed to achieve the desired level of gene expression over a period of various intervals, e.g., daily, weekly, monthly, yearly, etc.

Injectables can be prepared in conventional forms, either as liquid solutions or suspensions, solid forms suitable for solution or suspension in liquid prior to injection, or as emulsions. Alternatively, one may administer the virus vector and/or virus capsids of the invention in a local rather than systemic manner, for example, in a depot or sustained-release formulation. Further, the virus vector and/or virus capsid can be delivered adhered to a surgically implantable matrix (e.g., as described in U.S. Patent Publication No. US-2004-0013645-A1).

Disorders of the CNS include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The delivery vectors of the present invention can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly.

Uveitis involves inflammation. One or more anti-inflammatory factors can be administered by intraocular (e.g., vitreous or anterior chamber) administration of a delivery vector of the invention.

Retinitis pigmentosa, by comparison, is characterized by retinal degeneration. In representative embodiments, retinitis pigmentosa can be treated by intraocular (e.g., vitreal administration) of a delivery vector encoding one or more neurotrophic factors.

Age-related macular degeneration involves both angiogenesis and retinal degeneration. This disorder can be treated by administering the inventive deliver vectors encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the inventive delivery vectors. Such agents include N-methyl-D-aspartate (NMDA) antagonists, cytokines, and neurotrophic factors, delivered intraocularly, optionally intravitreally.

In particular embodiments, the vector can comprise a secretory signal as described in U.S. Pat. No. 7,071,172.

The virus vector and/or capsid may also be administered to different regions of the eye such as the retina, cornea and/or optic nerve.

In particular embodiments, the virus vector and/or capsid is administered in a liquid formulation by direct injection (e.g., stereotactic injection) to the desired region or compartment in the CNS. In other embodiments, the virus vector and/or capsid may be provided by topical application to the desired region or by intra-nasal administration of an aerosol formulation. Administration to the eye, may be by topical application of liquid droplets. As a further alternative, the virus vector and/or capsid may be administered as a solid, slow-release formulation (see, e.g., U.S. Pat. No. 7,201,898).

Having described the present invention, the same will be explained in greater detail in the following examples, which are included herein for illustration purposes only, and which are not intended to be limiting to the invention.

### EXAMPLES

### Example 1

Recombinant adeno-associated virus (rAAV) has become the preferred vector for retinal gene transfer. Delivery of rAAV to the retina through the vitreous results in few serotypes efficiently transducing the retina. These few serotypes have capsid proteins which bind to heparan sulfate proteoglycan (HSPG). The interaction between capsid and receptor was evaluated using rAAV capsid with modified receptor interactions. Viruses were delivered intravitreally in adult mice and evaluated eight weeks later. Mutations in heparan sulfate (HS) binding residues of rAAV2 led to a dramatic decrease in the transduction of the inner retina. Elements of the non-HS binding rAAV1 were added to rAAV2 using the designer chimeric capsid, rAAV2.5. rAAV2.5 transduced along retinal vessels and showed greater expression in Muller glia cells. The addition of HS binding to rAAV1 showed an increase in fluorescence which resembled the expression pattern observed with rAAV2.5. The dual receptor interaction of HS and galactose was evaluated using a recently designed chimeric capsid, rAAV2G9, to determine if the addition of galactose binding would enhance capsid transduction. rAAV2G9 revealed an altered fluorescence pattern by fundus compared to rAAV2. The transduction profile of rAAV2G9 showed a shift in tropism to Muller glia. Taken together, HS binding is essential for successful intravitreal transduction, and this transduction can be skewed to specific retinal cells by the addition of galactose binding.

### Example 2. Heparan Sulfate Binding Promotes Accumulation of Intravitreally-Delivered Adeno-Associated Viral Vectors at the Retina for Enhanced Transduction

Many adeno-associated virus (AAV) serotypes efficiently transduce the retina when delivered to the subretinal space, but show limited success when delivered to the vitreous due to the inner limiting membrane (ILM). Subretinal delivery of rAAV2 and its HS-binding-deficient capsid indicated rAAV2 transduction of the outer retina occurred by HS-independent mechanisms. However, intravitreal delivery of HS-ablated rAAV2 lead to a 300-fold decrease in transduction compared to rAAV2. Fluorescence in situ hybridization (FISH) of AAV retinal trafficking revealed a mechanism of AAV2 accumulation at the ILM was influenced by HS binding. This mechanism was tested on human ex vivo retinas and showed similar accumulation only with HS-binding AAV2 capsid. To evaluate if HS binding could be applied to other AAV serotypes to enhance their transduction, AAV1 and AAV8 were modified to bind HS with a single amino acid mutation and tested in mice. Both HS-binding mutants of AAV1 and AAV8 had higher intravitreal transduction over their non-HS-binding parent capsid. To understand the influence HS binding has on AAV2 tropism, chimeric capsids with dual glycan usage were tested intravitreally in mice. Compared to HS binding alone, these chimeric capsids displayed enhanced transduction that was correlated to a change in tropism. Taken together, this indicates that HS-binding serves to sequester AAV capsids from the vitreous to the ILM, but does not influence tropism. The enhanced retinal transduction of HS-binding capsids provides a rational design strategy for engineering capsids across species for intravitreal delivery.

The present studies show that HSPG binding is correlated in greater accumulation and transduction in the retina. We validated that this accumulation is conserved in mouse and human retinas. The addition of HSPG binding to any AAV capsid can increase the number of serotypes which show efficient intravitreal transduction.

Adeno-associated virus (AAV) is a small (25 nm), non-pathogenic virus that has been extensively studied as a vector for gene transfer applications. The virus consists of two parts: the viral genome and the protein capsid. The viral genome can be largely replaced with a desired transgene to create recombinant AAV (rAAV) vectors used for gene delivery. The protein capsid is responsible for cell attachment and entry via a variety of glycans and cell surface receptors. There exist eleven naturally-occurring serotypes of AAV, denoted as AAV1 to AAV11. Glycans and receptors have been elucidated for several AAV serotypes. Heparan sulfate proteoglycan (HSPG) has been shown to be used for both rAAV2 and rAAV3 cell entry. rAAV6 displays dual glycan interaction with HSPG and sialic acid; however, HSPG binding alone is insufficient for cellular entry. Various linkages of sialic acid are important for the transduction of rAAV1, rAAV4, and rAAV5 serotypes. N-linked galactose is used for the transduction of rAAV9 serotype. Glycans expressed on the cell surface dictate the tissue and cellular tropism observed with the various AAV capsids. In addition to the attachment to these glycans, AAV serotypes interact with cell receptors for entry, including human growth factor receptor, integrins, and laminin receptors.

rAAV has shown promise for retinal gene transfer. In addition to the influence of the capsid as discussed above, the route of administration to the retina determines the transgene expression profile and efficacy. Subretinal (SR) delivery deposits vector between the outer nuclear layer (ONL) and retinal pigment epithelium (RPE), which causes a detachment of these two layers to accommodate the injected solution. Many serotypes display transduction in the ONL and RPE layers with some serotypes showing restricted tropism. Of the natural serotypes, rAAV8 is one of the best for SR delivery based on its rapid transgene expression and transduction of all retinal layers. In addition to SR delivery, vectors can be administered to the vitreous. Intravitreal delivery of rAAV vectors has become the preferred route to subretinal for several reasons, including 1) technical ease of injection, 2) the potential to deliver vector to a greater area of the retina, and 3) it's less damaging to the retina. For clinical applications, intravitreal delivery could be performed as an outpatient procedure and circumvent the retinal disruption may exclude applicable to patients with severe retinal degeneration. However, few serotypes exhibit efficient transduction by this route. rAAV2 is one of the few serotypes tested in multiple animal models, typically resulting in the transduction of retinal ganglion cells (RGC). In rodent models, transduction with this serotype has been seen in occasional Müller glia, amacrine, and horizontal cells. In addition, rAAV6 expression in the RGC and inner nuclear layer (INL) has been seen in rodent models. Understanding viral trafficking and barriers to efficient intravitreal transduction provides opportunities to rationally design capsids to overcome current limitations.

At the vitreoretinal junction, the inner limiting membrane (ILM) has been implicated as the barrier responsible for the inefficiency of most rAAV vectors to transduce the retina. Despite the limited transduction, several AAV serotypes are capable of accumulating at the vitreoretinal junction following delivery. Injections of fluorescently-labeled capsids (rAAV1, 2, 5, 8, and 9) into the vitreous of adult rodents showed rAAV2, rAAV8, and rAAV9 accumulated at the ILM, but only rAAV2 resulted in transduction. With a degenerated ILM, all of these AAV serotypes were capable of transducing the retina. The ILM is composed of the extracellular matrix of the Müller glial endfeet which displays an array of glycans similar to other basement membranes and prevents access to cells needed for AAV transduction. The binding of rAAV2, rAAV8, and rAAV9 is likely explained by laminin interaction; however, accumulation by laminin is not sufficient for transduction of rAAV8 and rAAV9. HSPG seems to explain the rAAV2 transduction, but enzymatic digestion of HSPG increases transduction and penetration of rAAV2 in the retina. Because rAAV2 has shown HSPG-independent transduction in other tissue, it is possible that rAAV2 does not need HSPG binding for retinal transduction and that HSPG may prevent the spread of rAAV2 particles to the outer retina. To this end, rAAV2 capsid interactions with HSPG at the ILM pose the rate-limiting step to efficient intravitreal transduction of the retina and understanding these interactions will help guide rational design of vectors for more efficient intravitreal delivery.

We used a self-complementary CBh-GFP cassette for optimized transgene performance. The CBh promoter has shown exceptional activity in other neuronal tissue compared to CMV or CBA promoter activity without potential silencing issues, and its small size is beneficial for maximizing the limited transgene capacity of rAAV. The self-complementary form of the transgene facilitates faster expression that is more robust than the classic single-stranded form. This self-complementary form can also facilitate production of transgene product in cells that do not provide second-strand synthesis. In addition to optimizing GFP production, we used FISH to track rAAV capsids following intravitreal delivery and obtain an accurate picture of the trafficking. Genetic capsid mutations were used to understand the role of HS binding to rAAV transduction of the mouse retina without modifying the ILM structure. We used known capsids mutations in the HS-binding footprint of rAAV2 to ablate HS binding. The motif on the rAAV2 capsid consists of a basic patch of residues (R484, R487, K532, R585, and R588) at the base of the three-fold spike. Capsid mutants, like rAAV2i8, replace residues 585Q and 588T to alter tropism away from HS-rich liver tissue and become more systemic when delivered intravenously. Using this capsid, we investigated the unilateral necessity for HS binding for retinal transduction.

Vector production and purification. Self-complementary rAAV carrying the GFP gene under the control of the ubiquitous CBh promoter was produced by a triple transfection method using polyethylenimine. Viruses were harvested. Lysate was clarified by centrifugation at 6200×g and purified by iodixanol gradient ultracentrifugation at 402,000×g for 1 hour. Viruses were pulled from the 40%/60% interface, purified by ion-exchange chromatography on a 1-ml Q HyperD F column (Pall) and eluted with 200 mM NaC1, 25 mM Tris [pH 9.0]. AAV8-E533K vector was difficult to produce in significant yield by iodixanol. Therefore, AAV8 and AAV8-E533K were purified by CsCl and then by sucrose to obtain pure vector. Viruses were dialyzed against 350 mM NaCl, 5% sorbitol in 1×PBS before being aliquoted and frozen at −80° C. Viral titer was determined by qPCR against wild-type ITR of DNase-resistant vector genomes relative to a virus standard. Viruses underwent electrophoresis on a 1% Bis-Tris gel (Novex) and silver stain (Life Technologies) to assess purity.

Animal injections. Adult C57BL/6 mice were used for this study. All animals were housed under 12/12 hours light/dark cycle in the University of North Carolina Division of Laboratory Animal Medicine facilities and were handled in accordance within the guidelines of the Institutional Animal Care and Use Committee at the University of North Carolina. Prior to vector delivery, animals were anesthetized with ketamine (75 mg/kg), xylazine (10 mg/kg), acepromazine (1.5 mg/kg), and dilated with 1% tropicamide and 2.5% phenylephrine. Proparacaine-HCl was applied to eyes as a local anesthetic. Intraocular needles were constructed using 32G canula connected to a Hamilton syringe via tubing filled with water. An air bubble separated the water from the viral suspension. Freshly thawed viruses were diluted to working stock and incubated in the intraocular needle at room temperature for 10 minutes prior to injection. Needles were evacuated and loaded with fresh suspension. Viral suspension was mixed with fluorescein sodium salt (Sigma) to confirm successful injection. All injections were carried out by the same surgeon. For intravitreal injections, a pilot hole was made with the tip of a beveled 30G needle in the superior portion of the eye approximately 0.5 mm posterior to the limbus. The intraocular needle was inserted through this hole into the vitreous under direct observation through the microscope. A volume of 1 microliter was delivered at a constant rate over 30 seconds using a syringe pump. The needle was held in place for 20 seconds to allow for intraocular pressure equilibration before removal. For subretinal injection, the intraocular needle was inserted tangential to the eye. Delivery of fluid was immediate and characterized for success by optical coherence tomography (OCT) and fundoscopy using the Micron IV (Phoenix Research Laboratories). GENTEAL eye drops were applied to eyes to prevent corneal drying, and mice were allowed to recover on heating pads.

In vivo imaging. Fundus images and OCT were carried out by dilating and sedating animals as described herein. All fluorescence images were taken under the same settings and similar retinal position using the Micron IV. The green channel of the fluorescent fundus image was isolated, converted to grayscale, and quantified by integrated density measurements using ImageJ software (National Institutes of Health).

Enucleation and histology. Animals were sedated and perfused with PBS containing 1 unit heparin per ml, followed by 4% paraformaldehyde (PFA) in PBS. Eyes were enucleated and a puncture was made anterior to the limbus using an 18G needle before incubating in 4% PFA for 10 minutes. The anterior segment, musculature, and lens were removed and eyecups were placed in 10% sucrose at 4° C. overnight followed by 20% and 30% sucrose incubations. Eyecups were embedded in OCT cutting media (Sakura), frozen at −20° C., and stored at −80° C. Ten micron transverse sections were collected on precleaned Superfrost Plus slides (Fisher) and stored at −80° C. until further processing.

Immunohistochemistry and analysis. Sections were washed in TBS containing 0.3% Tween-20 (TBS-T) and incubated in blocking buffer (10% normal goat serum, 0.1% Triton-x 100 in PBS) for 1 hour in a humid chamber. Slides were incubated in antibody solution (3% NGS, 0.1% TRITON-X100 in PBS) with primary antibodies in a humid chamber overnight at room temperature. Primary antibodies used were rabbit anti-GFP (1:500; Millipore), mouse anti-glutamine synthetase (1:100; Abcam), mouse anti-heparan sulfate 10E4 epitope (1:70; Amsbio), mouse anti-rhodopsin (1:100; Rockland), and mouse anti-PKC alpha [H-7] (1:250; Santa Cruz). After three washes with TBS-T, secondary fluorescent antibodies were applied in antibody solution for 2 hours in a humid chamber. Secondary antibodies were Alexa-Fluor 488 goat anti-rabbit (1:1000; Molecular Probes), Alexa-Fluor 568 goat anti-mouse (1:1000; Molecular Probes), or Alexa Fluor 568 rabbit anti-goat (1:1000; Molecular Probes). Slides were mounted in Prolong Gold Antifade with DAPI (Molecular Probes) as stated by manufacturer's protocol. Images were taken on a LeicaSP2

AOBS Upright Laser Scanning Confocal microscope or Olympus IX83 fluorescence microscope.

Soluble HS analog assays. For in vitro studies, HEK293 cells were plated in a 24-well dish at a density of 10⁵ cells per well and allowed to adhere overnight at 37° C., 5% CO₂. Viruses were pre-incubated with soluble heparin at the specified concentrations for 1 hour prior to the addition to cells at a concentration of 10,000 vg per cell. Cells were harvested 48 hours later and quantified by flow cytometry.

Fluorescence in situ hybridization. The GFP gene was cloned into the pSPT18 vector (Roche RNA in vitro transcription kit) at the HindIII and EcoRI sites and sequenced for confirmation. Plasmids were linearized with these restriction enzymes and purified by phenol-chloroform extraction/ethanol precipitation and resuspended in water. Linearized plasmids were quantified by spectrophotometry and verified by gel electrophoresis before in vitro transcription of antisense and sense riboprobes were carried as described by the manufacturer (Roche). Aliquots of riboprobes were frozen in water and were quantified as described by the manufacturer and analyzed by gel electrophoresis and SYBR Gold staining

(Invitrogen). Riboprobe functionality was assayed for sensitivity and selectivity by dot blot of virus controls to a positively-charged nitrocellulose membrane (Roche). Both sense and antisense probe were able to detect viral GFP transgene equally.

Frozen slides were heated to 55° C. for 10 minutes and pre-treated. Slides were then incubated in hybridization buffer (50% formamide, 10 mM Tris [pH 7.6], 200 μg/ml yeast tRNA, 1×Denhardt's solution, 10% dextran sulfate, 600 mM NaCl, 0.25% SDS, 1 mM EDTA [pH 8]) without probe at hybridization temperature of 65° C. for 2 to 4 hours. Slides were transferred to prehybridization buffer containing 50 ng/ml of sense riboprobe to specifically detect DNA and not mRNA transcripts. Slides were heated to 80° C. for 20 minutes, snap chilled on ice, and incubated overnight at 65° C. Slides were washed in 50% formamide/2×SSC at 65° C. for 30 minutes, 2×SSC at 55° C. for 20 minutes, and two washes of 0.2×SSC at 55° C. for 20 minutes. Slides were washed in 1×Washing Buffer (Roche) followed by incubation of 10% sheep serum in 1×Blocking Buffer for 1 hour in a humid chamber. Sheep anti-DIG-AP antibody (1:1000; Roche) was applied and incubated 2 to 3 hours in a humid chamber. Slides were washed three times in Washing Buffer with gentle agitation for 10 minutes followed by two incubations in Detection Buffer (100 mM Tris, 100 mM NaCl, 10 mM MgCl₂ [pH 8.0]) for 10 minutes. HNPP/Fast Red detection substrate was prepared and applied as directed by the manufacturer (Roche) for two to three applications. Following the detection reaction, slides were rinsed in distilled water and coverslipped using Prolong Gold antifade reagent with DAPI. Images were taken on a LeicaSP2 AOBS Upright Laser Scanning Confocal microscope or an Olympus IX83 fluorescence microscope.

Ex vivo human retinal binding assay. Human whole globes were procured immediately after death and placed in a moist chamber on ice for 4 days. The anterior chamber, iris, and lens were removed and the globe was quartered with some vitreous remaining attached to the retina. Ten μl of vector was applied into the vitreous at a titer of 2×10⁹ vg/μl and allowed to bind to the retina for 2 hours at 4° C. The quartered retinas were kept out of media to prevent the dispersion of vector solution. Following the incubation, PBS was washed over the tissue, collected, and stored at −80° C. Vitreous, retina, choroid, and sclera were collected separately and stored at −80° C. Tissue samples were digested and purified using the DNeasy Blood & Tissue kit (Qiagen). Virus in the collected samples was quantified by qPCR using primers against GFP and hGAPDH housekeeping genes.

HS binding on rAAV2 is not required for subretinal transduction. A variety of AAV serotypes, both HS binding and non-HS binding, work effectively in retinal transduction when delivered subretinally. To determine if rAAV2 requires HS binding in the transduction of the outer retina, HS-deficient rAAV218 and rAAV2 were subretinally delivered. Transduction between them was similar by fundoscopy. The strongest signal of the GFP fluorescence with rAAV218 was seen within the detached area but expression could be seen outside the bleb area. Similarly, rAAV2-injected eyes showed that transduction occurred mainly in the area of the detachment. Both vectors resulted in large areas of transduction that appeared to be the RPE. For rAAV2, transduction of ganglion cells was evident by the fluorescent axons leading to the optic head from the site of injection.

Immunohistochemistry (IHC) was used to evaluate the cell tropism of both vectors. The RPE and ONL were the major cell layers transduced by both vectors. Areas could be seen where high RPE transduction but low ONL transduction occurred, indicating that RPE may be the predominant cell type to be transduced. Transduction of the ONL occurred predominately in rods for both rAAV2 and rAAV218 capsids. rAAV218 transduction of cells in the INL was identified as rod bipolar cells and Müller glia. These results confirm that HS-deficient rAAV2 capsid is infectious in the retina by subretinal delivery.

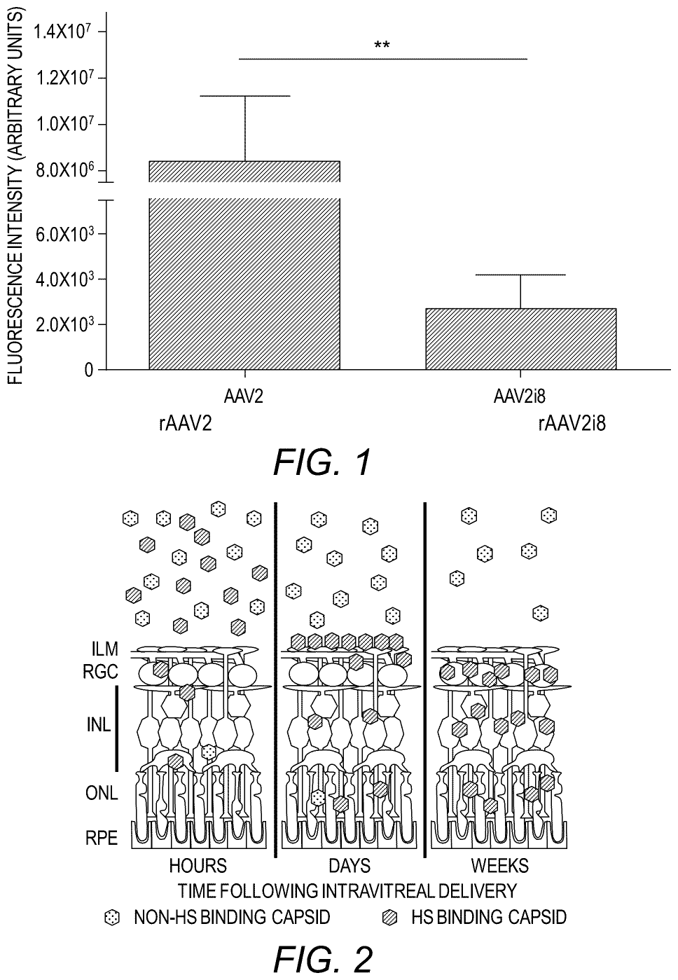

HS binding of rAAV2 is required for intravitreal transduction. To assess whether HS-binding is necessary for intravitreal transduction, AAV2 and AAV218 capsids were delivered to adult mice at a titer of 108 vg. rAAV2-injected eyes were fluorescent at the first imaging time point of two weeks whereas rAAV2i8 showed no expression. Eyes were evaluated for up to twelve weeks for the possibility of slower expression kinetics. During that time, rAAV2 fluorescence continued to increase, but no fluorescence was detected with rAAV2i8. By twelve weeks, rAAV2 capsid leads to a diffuse pattern of fluorescence over the neural retina as seen by fundus imaging. The rAAV218 capsid did not yield observable GFP fluorescence by fundoscopy and resulted in a 300-fold reduction in GFP fluorescence (FIG. 1). We confirmed that the vitreous was not inhibitory to the transduction of the HS-ablated rAAV2 capsid by mixing vitreous and virus before injecting subretinally. Again, rAAV2 and rAAV218 had intense expression throughout most of the retina observed on fundoscopy. The transduced cells for both capsids appeared to be RPE, but the addition of RGCs can be seen with rAAV2.

Eyes injected intravitreally with rAAV were further evaluated by IHC. rAAV2 transduction was mainly detected in the RGC and INL, and in some sections, transduction of photoreceptors could be observed. The histology of HS-ablated rAAV2 capsids revealed very few GFP-positive rods, but most of the retina remained negative. We tried a higher titer of 2×10⁹ vg for both rAAV2 and its HS-binding mutant to maximize the chance to observe expression. The expression was much greater with higher titered rAAV2 virus compared to what was observed with the lower titer-injected eyes, but the pattern of transduction was unchanged.

Fluorescence in situ hybridization (FISH) was used to determine the distribution of transgenes between the two capsids after intravitreal delivery. Studies of subretinally-injected virus have shown that virus distribution and transgene expression are not synonymous; therefore, we wanted to determine how HS binding affected rAAV2distribution irrespective of expression. Similar to the IHC expression, FISH signal for rAAV2-delivered transgenes was detected mainly in the RGC and INL, with fewer transgenes in the ONL. rAAV218-injected eyes showed GFP transgenes present in the ONL, but none were detected in the RGC or INL. Because histology was performed months after injection, these transgenes most likely represent episomes that are stable following entry into the retina by intravitreal delivery.

HS binding is necessary for the vitreal accumulation of rAAV2 at the ILM in mice. To better understand the trafficking differences between the capsids following intravitreal delivery, eyes were enucleated soon after injection for FISH analysis. Because we have established that exposure of charged residues on the capsid surface are important in transduction, we used FISH as an alternative to modifying the capsid with fluorescent particles for trafficking experiments. That AAV particles accumulate at the ILM 24 hours post-injection was confirmed using our FISH protocol and this indicated we could detect transgenes carried by capsids that were still intact. The time point was extended to three days post-injection to allow sufficient time for capsid accumulation at the ILM and observe trafficking differences between the rAAV2 capsid and its HS-binding mutants. A range of doses was used to capture any concentration effect in accumulation and the enzymatic time for FISH signal was shortened to give a more dynamic range. PBS-injected eyes served as the negative control which had minimal background labeling. With the shortened detection time, a dose of 1×10⁸ vg had only weak signal in retinas for both rAAV2 and HS-deficient rAAV2-R585E capsids. At a dose of 5×10⁸ vg, transgenes delivered by rAAV2 showed an accumulation at the ILM, as well as being present in all retinal layers. Without HS binding, rAAV2-R585E had only minimal signal. At the highest dose tested of 2×10⁹ vg, rAAV2 resulted in even greater signal intensity at the ILM with sporadic transgenes detected in multiple retinal layers. At the same dose, few rAAV2-R585E-delivered transgenes were detected in the retina but did not result in any accumulation at the ILM.

Taken together, these results indicate 1) that HS binding on rAAV2 helps to accumulate vectors at the ILM, 2) that this accumulation increases the number of transgenes residing in the retina, 3) that capsids can penetrate the retina from the vitreous without binding to HS but to a far less extent, and 4) that HS binding is not required for rAAV2 transduction of the retina when vector is delivered subretinally. The FISH data indicate that of the capsids that quickly pass through the ILM barrier, they seem capable of trafficking rapidly to distal layers of the outer retina (FIG. 2). This highlights that rAAV's rate-limiting step to efficient intravitreal transduction of the retina lies with the interaction between capsid and ILM.

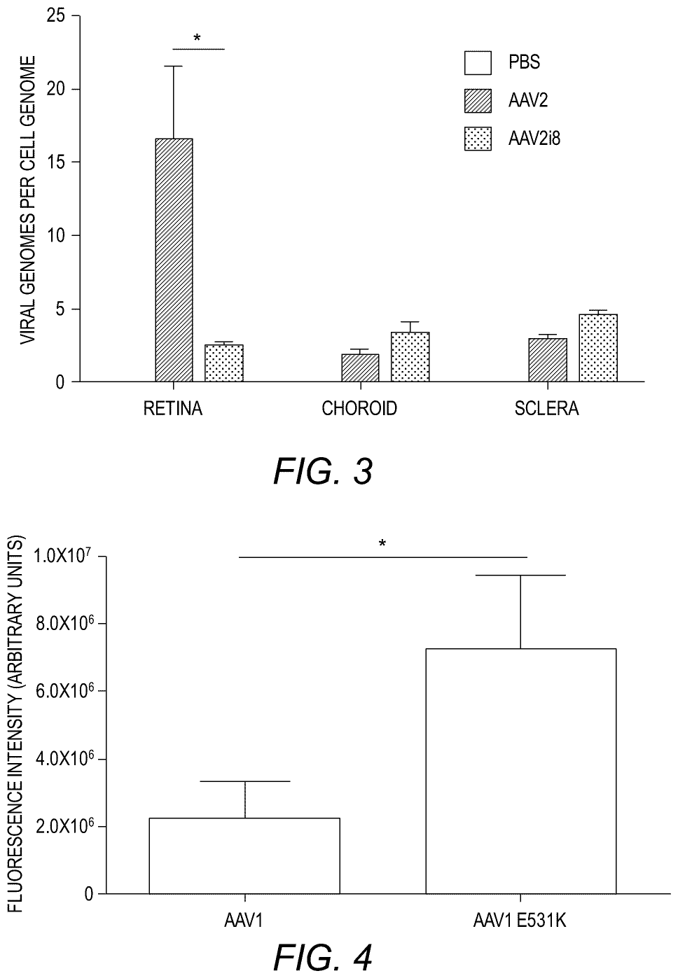

HS binding is necessary for the vitreal accumulation of rAAV2 on human retinas. The abundant HSPG staining at the ILM is present in many animal models, including humans. This suggests that this mechanism may translate across species for human clinical applications. A viral binding assay was done on human retinas ex vivo by quartering the eye and leaving a small amount of vitreous attached to the retina to maintain the ILM. Vectors were applied into the vitreous and then the various retinal layers were harvested. Transgenes carried by rAAV2 were bound to the retina, unlike those of the rAAV218 capsid. The HS-deficient rAAV218 had relatively low vector binding in any of the collected tissue, but did show a significant increase in binding to the choroid and sclera compared to rAAV2 (FIG. 3). Together with the mouse data, these results corroborate the mechanism of HS binding promoting the accumulation of AAV vector out of the vitreous and onto the ILM and later serves to enhance the transduction in the retina.

HS binding increases the intravitreal transduction of other rAAV serotypes. Intravitreal transduction of other serotypes may benefit from the addition of a HS-binding motif and carried out this selection in mice. The rAAV1 and rAAV6 serotypes differ by only six amino acids, with a single residue responsible for their difference in HSPG binding. To evaluate the influence of HS binding between rAAV1 and rAAV6 retinal transduction for intravitreal transduction, the single residue mutant capsids were tested intravitreally. Although rAAV1 and the HS binding rAAV1-E531K had similar patterns of expression, rAAV1-E531K had 3-fold greater GFP fluorescence compared to rAAV1 (FIG. 4). The removal of HS-binding in rAAV6 using the rAAV6-K531E capsid led to a reduction in retinal fluorescence by fundoscopy. Both rAAV1 and rAAV6 capsids displayed a punctate expression pattern around the retinal vessels. Because of the homology between rAAV1 and rAAV6, only rAAV1 and rAAV1-E531K were further evaluated for possible differences in cell tropism. Immunohistochemistry showed the transduction of mainly Müller glia for both capsids by the colocalization of GFP and glutamine synthetase, although additional cells of the INL appear to be transduced. In addition, both rAAV1 and rAAV1-E531K showed transduction of a few RGC and photoreceptors. The similar transduction patterns of rAAV1 and rAAV1-E531K indicate that HS-binding has not altered the tropism of rAAV1.

To confirm that the HS-binding mutation on rAAV1 does not convey use of HSPG for transduction, soluble heparin was mixed with capsids and applied to cells for an in vitro competition assay. rAAV2 requires HSPG for in vitro transduction and showed a dose dependent decrease in transduction. Neither rAAV1 nor rAAV1-E531K transduction were affected at any heparin dose, indicating that the rAAV1-E531K capsid does not depend on HS binding for transduction (FIG. 6). On average, transduction with rAAV1-E531K led to fewer GFP-positive cells compared to rAAV1, indicating that the single amino acid change alone does not provide an enhancement in transduction. A single amino acid mutant has been identified on rAAV8 providing HS-binding ability. Titers of rAAV8 and rAAV8-E533K were matched to 1×10⁸ vg and injected intravitreally. At eight weeks post-administration, fundus images were taken of the injected eyes. Transduction of rAAV8 was very low. Eyes injected with rAAV8-E533K resulted in hazy fluorescence over the retina, which when quantified, was higher compared to the non-binding serotype. We used FISH analysis to observe trafficking differences soon after injection and found again that HS-binding promoted the accumulation of vector at the ILM and within the retina. These results suggest that HS binding alone is sufficient to enhance the transduction of intravitreally-delivered AAV capsids by increasing the amount of vector that accumulate onto the retina.

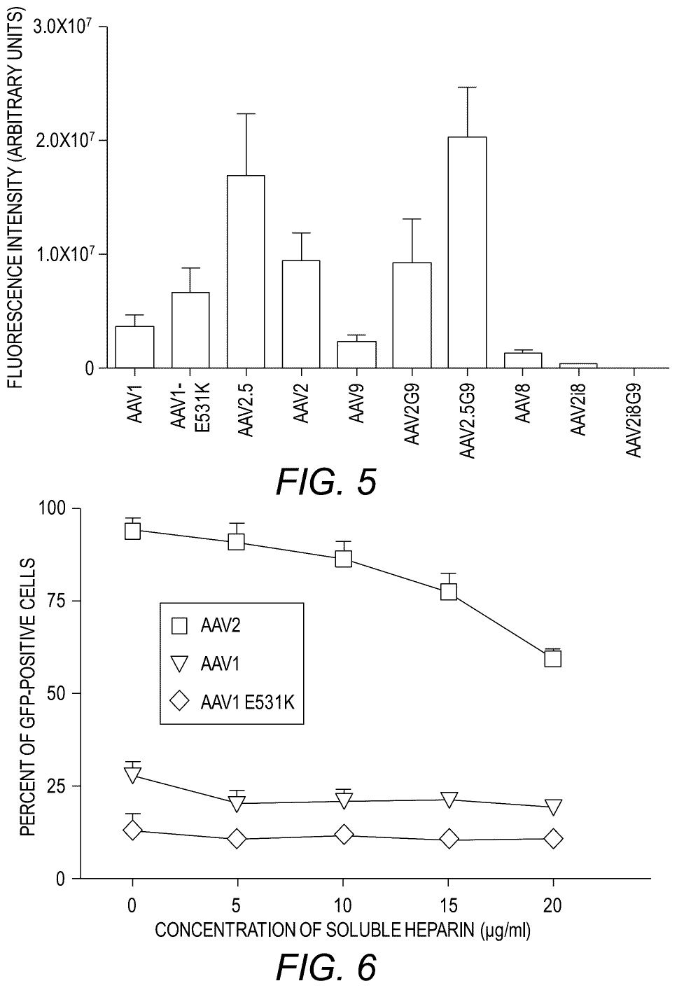

A double chimera of a rAAV2.5G9 capsid was used to determine which of rAAV1 or rAAV9 capsid elements were dominant. The intravitreal delivery of rAAV2.5G9 led to expression similar to the rAAV2.5 parent when imaged by fundoscopy. The fluorescence around the retinal vessels was very evident with punctate expression found around the vessels. Quantification of this expression when compared all together showed rAAV2.5 and rAAV2.5G9 to have the highest transduction and non-HS-binding capsids showing the lowest transduction (FIG. 5).

All together, the binding to HSPG at the ILM promotes rAAV accumulation from the vitreous onto the retina, but motifs from other non-HS-binding capsids can be used to influence retinal transduction patterns. This would explain how rAAV1-E531K maintained the transduction profile of rAAV1 despite having HS-binding. It would be difficult to accurately compare rAAV2 to rAAV1-E531K as these two capsids differ in residue composition and affinity to HS. Therefore, to assess the influence that rAAV1 can have on intravitreal transduction, we used the rAAV2.5 chimeric capsid. This capsid showed greater transduction than either parent and can be skewed to transduce Müller glia by the addition of galactose binding. Although the retinal glycan staining does not readily suggest the tropism, these chimeric capsids are reagents for targeted transduction in the retina. Because HSPG is abundant at the ILM in several animal models, this mechanism could be applied across multiple species, including humans. Once at the ILM, other glycan interactions with the rAAV capsid promote the tropism profile observed with the various capsid mutants.

The interaction of rAAV capsids with the ILM poses the rate-limiting step to retinal trafficking. Through the use of FISH, a small number of capsids pass through the ILM and traffic to the ONL and outer segments rapidly. Once in the outer retina, these vectors can transduce the photoreceptors but this transduction is very rare. Perhaps the majority of the capsids that enter the retina do not successfully traffic to the nucleus once in the cell to establish latency. Increasing the viral concentration is more likely to evoke an immune response as the vitreal space is not immune privileged like the subretinal space. The number of transgenes making it to the nucleus could be increased by using a higher titer. This enhanced intracellular trafficking may help to increase the transgene expression of intravitreally-delivered rAAV218 and rAAV2-R585E capsids. Accumulation at the ILM can be a function of vector concentration in that high doses of vector can lead to the increased transgenes found at the ILM and in the retina. To take advantage of the retinal structure, binding to the ILM can be used to accumulate vectors at the retina for transduction. This is likely why rAAV2 has been successful at intravitreal retinal transduction.

The charged sulfate groups facilitate the interaction of HSPG with the rAAV2 capsid for transduction. The transgenes observed by FISH indicate that HS-deficient rAAV2 particles are able to traffic to distal layers of the retina, which indicate that these charged residue changes on the capsid do not prevent the vector from entering the retina, but just limit the number of vectors accumulating on the retina. This is also demonstrated by the rare transduction of rods with HS-deficient capsid. To test if the capsid interaction with the sulfate is necessary, we mixed rAAV2 with sulfated and unsulfated HS and tested intravitreally. We found both forms could inhibit transduction with the sulfated heparin leading to greater inhibition. This experiment only suggests the importance of the interaction with the heparan chain to intravitreal transduction. The genetic capsid mutants seem to validate this chemical inhibition assay, but again, the single and double amino acid modifications that disrupt the basic patch of residues involved in HSPG binding rely on a charge interaction. Nanoparticles coated with specific charged formulations indicated cationic charges (basic) to be successful in promoting penetration in the retina from the vitreous. This may help to explain how glycans present at the ONL can influence the transduction of intravitreally delivered rAAV vectors.

The similar transduction profiles of SR-delivered rAAV2 and rAAV218 indicate that rAAV2 does not require HS binding for retinal transduction. It may be that the subretinal delivery effectively concentrates the vector, thereby stoichiometrically skewing capsids towards expression. In addition to transduction by receptor-mediated endocytosis, SR provides abundant rAAV vectors to the phagocytic RPE and may explain why the RPE appears to be the primary cell target by both rAAV2 and rAAV2i8. Regardless of the affinity towards any particular cell type, rAAV2 and rAAV2i8 vectors lead to transduction of RPE, rods, cones, rod bipolar cells, and Müller glia. The majority of these transduced cells are located within the injection bleb, but transduction of the RPE can be seen far outside the detached area. FISH could be used to map the trafficking of SR-delivered vector.

The ILM structure is found across multiple species and could serve to attract and concentrate rAAV capsids out of the vitreous. Indeed, HS binding led to a greater presence of transgenes in the retina compared to the parent capsid when assessed by FISH soon after injection. Transgenes of non-HS-binding rAAV1 and rAAV8 could still be detected in the retina, but to a less extent, similar to the data observed between rAAV2 and rAAV2-R585E capsids. The lack of expression with HS-binding rAAV3 when injected intravitreally was expected because this serotype is inefficient in the transduction of most cell types and may encounter additional barriers for efficient transduction of cells. rAAV6 is a serotype of interest for retinal transduction and has been modified to increase the specific transduction of Müller glia using the ShH10 capsid. Without HS binding, the ShH10 vector may reveal a much weaker fluorescence. Because both rAAV6 and rAAV1-E531K do not rely on HSPG-mediated transduction, simply adding the ability to bind to HS to any capsid serotype could enhance its transgene expression. We tested this by using rAAV8-E533K mutant capsid. The addition of HS binding could be applied to other serotypes for a greater breath of AAV used for intravitreal delivery.

Although HSPG is abundant at the ILM, other receptors can play a role in the transduction of the retina from the vitreous. The transduction by rAAV1 and rAAV6 suggest the presence of 2,3-or 2,6-N-linked sialic acid at the ILM despite the lack of staining in that region. The pattern of transduction observed by fundus may indicate a distinct pattern of this sialic acid in the retina that is not visible by histology by could be seen by flat mount. Other forms of sialic acid known to interact with rAAV4 and rAAV5 may not be expressed abundantly at the ILM or could be masked by other glycans. This would explain the lack of transduction by these serotypes when compared in a normal mouse retina. In addition to sialic acid, laminin staining is abundant at the ILM and restricted to the blood vessels. Laminin receptor is known to interact with rAAV2, rAAV3, rAAV8, and rAAV9 serotypes. Although these capsids can interact with laminin receptors at the vitreoretinal junction, this interaction seems insufficient to promote efficient intravitreal retinal transduction.

It is important to remember that the transduction observed in a mouse model may not be indicative to other models. While the mouse has become a standard model for retinal gene transfer, certain size and anatomical differences exist between them and primates. Other models, such as rabbit or pig, have similarly sized globes and vitreal volumes compared to primates. In addition, the thickness differences of the ILM between mouse and primates may lead to selection of capsids that are not efficient across species. The over abundance of HSPG at the ILM in multiple animal species, including avian, rodent, rabbit, primate, and human makes studying the influence of capsid interaction with HS important for the rational design of efficient vectors for intravitreal retinal gene transfer.

The foregoing is illustrative of the present invention, and is not to be construed as limiting thereof. The invention is defined by the following claims, with equivalents of the claims to be included therein.

All publications, patent applications, patents, sequences and other references mentioned herein are incorporated by reference herein in their entirety.

TABLE 1

| | GenBank Accession Number |
|---|---|
| Complete Genomes | |
| Adeno-associated virus 1 | NC_002077, AF063497 |
| Adeno-associated virus 2 | NC_001401 |
| Adeno-associated virus 3 | NC_001729 |
| Adeno-associated virus 3B | NC_001863 |
| Adeno-associated virus 4 | NC_001829 |
| Adeno-associated virus 5 | Y18065, AF085716 |
| Adeno-associated virus 6 | NC_001862 |
| Avian AAV ATCC VR-865 | AY186198, AY629583, NC_004828 |
| Avian AAV strain DA-1 | NC_006263, AY629583 |
| Bovine AAV | NC_005889, AY388617, AAR26465 |
| AAV11 | AAT46339, AY631966 |
| AAV12 | ABI16639, DQ813647 |
| Clade A | |
| AAV1 | NC_002077, AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu. 19 | AY530584 |
| Hu. 20 | AY530586 |
| Hu 23 | AY530589 |
| Hu22 | AY530588 |
| Hu24 | AY530590 |
| Hu21 | AY530587 |
| Hu27 | AY530592 |
| Hu28 | AY530593 |
| Hu 29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| Hu14 (AAV9) | AY530579 |
| Hu31 | AY530596 |
| Hu32 | AY530597 |
| Clonal Isolate | |
| AAV5 | Y18065, AF085716 |
| AAV 3 | NC_001729 |
| AAV 3B | NC_001863 |
| AAV4 | NC_001829 |
| Rh34 | AY243001 |
| Rh33 | AY243002 |
| Rh32 | AY243003 |

TABLE 2

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 3

| | |
|---|---|
| Modified Amino Acid Residue | Abbreviation |

Amino Acid Residue Derivatives

| | |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | aIle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

TABLE 4

| Serotype | Position 1 | Position 2 |
|---|---|---|
| AAV1 | A263X | T265X |
| AAV2 | Q263X | −265X |
| AAV3a | Q263X | −265X |
| AAV3b | Q263X | −265X |
| AAV4 | S257X | −259X |
| AAV5 | G253X | V255X |
| AAV6 | A263X | T265X |
| AAV7 | E264X | A266X |
| AAV8 | G264X | S266X |
| AAV9 | S263X | S265X |

Where, (X) → mutation to any amino acid
(−) → insertion of any amino acid
Note:
Position 2 inserts are indicated by the site of insertion

That which is claimed is:

1. A method of introducing a nucleic acid molecule into a retinal cell and/or a retinal pigment epithelium cell of a subject, comprising intravitreally administering to the subject an adeno-associated virus (AAV) vector comprising:
a) the nucleic acid molecule; and
b) a capsid protein of AAV4, AAV5, AAV7, AAV8, or AAV9, which comprises an amino acid substitution at position 530 of AAV4, position 517 of AAV5, position 533 of AAV7, position 533 of AAV8, or position 531 of AAV9, respectively, that increases heparan sulfate binding;
to thereby introduce the nucleic acid molecule into the cell.

2. The method of claim 1, wherein the amino acid substitution is a K.

3. The method of claim 2, wherein the AAV is AAV4.

4. The method of claim 2, wherein the AAV is AAV5.

5. The method of claim 2, wherein the AAV is AAV7.

6. The method of claim 2, wherein the AAV is AAV8.

7. The method of claim 2, wherein the AAV is AAV9.

8. The method of claim 1, wherein the nucleic acid molecule encodes a therapeutic protein or a therapeutic RNA.

9. The method of claim 1, wherein the subject is a human.

10. The method of claim 1, wherein the subject is a geriatric human.

11. A method of treating a disorder or a defect of the eye in a subject, comprising intravitreally administering to the subject an adeno-associated virus (AAV) vector comprising:
a) a capsid protein of AAV4, AAV5, AAV7, AAV8, or AAV9, which comprises an amino acid substitution at position 530 of AAV4, position 517 of AAV5, position 533 of AAV7, position 533 of AAV8, or position 531 of AAV9, respectively, that increases heparan sulfate binding; and
b) a nucleic acid molecule that encodes a therapeutic protein or a therapeutic RNA effective in treating the disorder or the defect of the eye, in expressible form;
to thereby treat the disorder or the defect.

12. The method of claim 11, wherein the disorder or the defect of the eye is age-related macular degeneration, Lebers congenital amarousis type 1, Lebers congenital amarousis type 2, retinitis pigmentosa, retinoschosis, achromatopsia, color blindness, congenital stationary night blindness, or any combination thereof.

13. The method of claim 11, wherein the subject is a human.

14. The method of claim 11, wherein the amino acid substitution is a K.

15. The method of claim 11, wherein the subject is a geriatric human.

16. The method of claim 11, wherein the disorder is an age-related disorder.


SEQUENCE LISTING (11 sequences)

SEQ ID NO: 1 - protein, 736 aa, Synthetic construct
  source 1..736: note = AAV1 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 735 aa, synthetic construct [sequence omitted]

SEQ ID NO: 3 - protein, 736 aa, synthetic construct
  source 1..736: note = AAV3 capsid
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 734 aa, synthetic construct
  source 1..734: note = AAV4 capsid
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 5 - protein, 724 aa, synthetic construct
  source 1..724: note = AAV5 capsid
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - protein, 737 aa, synthetic construct
  source 1..737: note = AAV7 capsid
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 7 - protein, 738 aa, synthetic construct
  source 1..738: note = AAV8 capsid
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 8 - protein, 675 aa, synthetic construct
  source 1..675: note = AAV9 capsid
MAADGYLPDLEDNLSEGIEWWALKPGAQPKANQQHQNARGLVLPGKYLGPGNGLDKGEPVNAADAALEHDKAYQQLKAGDNPLKYNHADAEQERLKEDTSGGNLGRAVFQAKKRLLEPLLVEEAAKTAGKKRPVEQSQEPDSSAGIKSGAQPAKKLNFGQTGDTESVPDPQPIGPPAAPSGVGLTMASGGGAVADNNEGADVGSSSGNWHDSQWLGDRVITTSTRTWALTYNNHLYKQSNSTSGGSSDNAYFGYSTWGYFDFNRFCHFSPRDWQRLINNNWGFRKRLNFKLFNQVKEVTDNNVKTIANNLTTVQVFTDSDQLPYVLGSAHEGCLPPFPAVFMIPQYGYTLNDGSQAVRSSFYCLEYPSQMLRTGNFQFSYEFENVPFHSSYAHSSLDRLMNPLDQYLYYLSKINGSGQNQQLKFSVAGPSMAVQGRNYIPGPSYRQQRVTTVTQNNNSFAWPGASSWLNGRNSLMNGPAMASHKEEDRFFPLSGSLIFGKQGTGDNVDADKVMTNEEEIKTTPVATESYGQATNHQSAQAAQTGWVQNQGILPGMVWQDDVYLQGPIWKIPHTDGNFPSPLMGGFGKHPPPQILINTPVPADPPTAFNKDKLNSITQYSTGQVVEIEWELQKNSKRWNPEIYTSNYYKSNVEFAVNTEGVYSEPRPIGTYLTRNL

SEQ ID NO: 9 - protein, 738 aa, synthetic construct
  source 1..738: note = AAV10 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 10 - protein, 733 aa, synthetic construct
  source 1..733: note = AAV11 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 11 - protein, 742 aa, synthetic construct
  source 1..742: note = AAV12 capsid
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL